SEQUENCE LISTING (46 sequences)

SEQ ID NO: 1 - protein, 36 aa, Artificial Sequence
  Human Immunodeficiency Virus Type I
SGIVQQQNNLLRAIEAQQHLLQLTVWGIKQLQARIL

SEQ ID NO: 2 - protein, 51 aa, Artificial Sequence
  Human Immunodeficiency Virus Type I
QARQLLSGIVQQQNNLLRAIEAQQHLLQLTVWGIKQLQARILAVERYLKDQ

SEQ ID NO: 3 - protein, 44 aa, Artificial Sequence
  Chimeric HIV-1 N38 GP41 Sequence
RGRGRGSGIVQQQNNLLRAIEAQQHLLQLTVWGIKQLQARILAG

SEQ ID NO: 4 - protein, 51 aa, Artificial Sequence; identical to SEQ ID NO: 2
  Human Immunodeficiency Virus Type I

SEQ ID NO: 5 - protein, 62 aa, Artificial Sequence
  Human Immunodeficiency Virus Type I
STMGAASMTLTVQARQLLSGIVQQQNNLLRAIEAQQHLLQLTVWGIKQLQARILAVERYLKD

SEQ ID NO: 6 - protein, 17 aa, Artificial Sequence
  Mutated N17 HIV-1 GP41 Sequence
LLQLTVWGIKALAAAIA

SEQ ID NO: 7 - protein, 16 aa, Artificial Sequence
  Mutated N17 HIV-1 GP41 Sequence
LIQLIWGIKQIQARIL

SEQ ID NO: 8 - protein, 51 aa, Artificial Sequence
  Mutated N51 HIV1 GP41 Sequence
QIRELISKIVEQINNILRAIEAQQHALQLTVWGIKQLQARILAVERYLKDQ

SEQ ID NO: 9 - protein, 51 aa, Artificial Sequence
  Mutated N51 HIV-1 GP41 Sequence
QARQLLSGIVQQQNNLLRAIEAQQHALQLTVWGIKQLQARILAVERYLKDQ

SEQ ID NO: 10 - protein, 51 aa, Artificial Sequence
  Mutated N51 HIV-1 GP41 Sequence
NIRQLLSGIVQQQNNLLRAIEAQQHLLQLTVWGIKQLQARILAVERYLKDQ

SEQ ID NO: 11 - protein, 51 aa, Artificial Sequence
  Mutated N51 HIV-1 GP41 Sequence
QARQLISGIVQQQNNLLRAIEAQQHLLQLTVWGIKQLQARILAVERYLKDQ

SEQ ID NO: 12 - protein, 51 aa, Artificial Sequence
  Mutated N51 HIV-1 GP41 Sequence
QIRQLISGIVQQQNNLLRAIEAQQHLLQLTVWGIKQLQARILAVERYLKDQ

SEQ ID NO: 13 - protein, 51 aa, Artificial Sequence
  Mutated N51 HIV-1 GP41 Sequence
QARQLLSAIVQQQNNLLRAIEAQQHLLQLTVWGIKQLQARILAVERYLKDQ

SEQ ID NO: 14 - protein, 51 aa, Artificial Sequence
  Mutated N51 HIV-1 GP41 Sequence
QIRQLISAIVQQQNNLLRAIEAQQHLLQLTVWGIKQLQARILAVERYLKDQ

SEQ ID NO: 15 - protein, 51 aa, Artificial Sequence
  Mutated N51 HIV-1 GP41 Sequence
QARQLLSGIVQQINNLLRAIEAQQHLLQLTVWGIKQLQARILAVERYLKDQ

SEQ ID NO: 16 - protein, 51 aa, Artificial Sequence
  Mutated N51 HIV-1 GP41 Sequence
NIRQLISAIVQQINNLLRAIEAQQHLLQLTVWGIKQLQARILAVERYLKDQ

SEQ ID NO: 17 - protein, 51 aa, Artificial Sequence
  Mutated N51 HIV-1 GP41 Sequence
QARQLLSGIVQQQNNLLRAIEAQQHLLQLVVWGIKQLQARILAVERYLKDQ

SEQ ID NO: 18 - protein, 51 aa, Artificial Sequence
  Mutated N51 HIV-1 GP41 Sequence
QARQLLSGIVQQQNNLLRAIEAQQHLLQLIVWGVKQLQARILAVERYLKDQ

SEQ ID NO: 19 - protein, 51 aa, Artificial Sequence
  Mutated N51 HIV-1 GP41 Sequence
QARQLLSGIVQQQNNLLRAIEAQQHLLQLTVWGIKQIQARILAVERYLKDQ

SEQ ID NO: 20 - protein, 51 aa, Artificial Sequence
  Mutated N51 HIV-1 GP41 Sequence
QARQLLSGIVQQQNNLLRAIEAQQHLLQLTVWGIKQLQARILAIERYIKDQ

SEQ ID NO: 21 - protein, 51 aa, Artificial Sequence
  Mutated N51 HIV-1 GP41 Sequence
QARQLLSGIVQQQNNLLRAIEAQQHLLQLTVWGIKQLQARILAVERYLKDI

SEQ ID NO: 22 - protein, 51 aa, Artificial Sequence
  Mutated N51 HIV-1 GP41 Sequence
QARQLLSGIVQQQNNLLRAIEAQQHLLQLIVWGVKQIQARILAIERYIKDI

SEQ ID NO: 23 - protein, 51 aa, Artificial Sequence
  Mutated N51 HIV-1 GP41 Sequence
QARQLLSGIVQQQNNLLRAIEAQQHLLQLVVWGNKQLQARVLAVERYLKDQ

SEQ ID NO: 24 - protein, 51 aa, Artificial Sequence
  Mutated N51 HIV-1 GP41 Sequence
QIRQLISGIVQQINNILRAIEAQQHLLQLIVWGIKQIQARILAIERYIKDQ

SEQ ID NO: 25 - protein, 48 aa, Artificial Sequence
  HIV Type I
ARQLLSGIVQQQNNLLRAIEAQQHLLQLTVWGIKQLQARILAVERYLK

SEQ ID NO: 26 - protein, 7 aa, Artificial Sequence
  HIV Type I
AVERYLK

SEQ ID NO: 27 - protein, 24 aa, Artificial Sequence
  HIV Type 1
LLQLTVWGIKQLQARILAVERYLK

SEQ ID NO: 28 - protein, 30 aa, Artificial Sequence
  HIV Type 1
IEAQQHLLQLTVWGIKQLQARILAVERYLK

SEQ ID NO: 29 - protein, 43 aa, Artificial Sequence
  HIV Type 1
SGIVQQQNNLLRAIEAQQHLLQLTVWGIKQLQARILAVERYLK

SEQ ID NO: 30 - protein, 5 aa, Artificial Sequence
  HIV Type I
ASQLL

SEQ ID NO: 31 - protein, 31 aa, Artificial Sequence
  Suzuki-1Z Coiled Coil Motif
YGGIEKKIEAIEKKIEAIEKKIEAIEKKIEA

SEQ ID NO: 32 - protein, 7 aa, Artificial Sequence
  Suzuki-1Z Coiled Coil Motif
IEKKIEA

SEQ ID NO: 33 - protein, 33 aa, Artificial Sequence
  1Z Domain
RMKQIEDKIEEILSKQYHIENEIARIKKLIGER

SEQ ID NO: 34 - protein, 23 aa, Artificial Sequence
  Shorted 1Z-Like Domain 1Z17
IKKEIEAIKKEEAIKKKIEAIEK

SEQ ID NO: 35 - protein, 17 aa, Artificial Sequence
  Chimeric N-Peptide 1ZN36
IKKEIEAIKKEQEAIKK

SEQ ID NO: 36 - protein, 62 aa, Artificial Sequence
  E7 Scaftold Domain
IKKEIEAIKKEQEAIKKKIEAIEKEISGIVQQQNNLLRAIEAQQHLLQLTVWGIKQLQARIL

SEQ ID NO: 37 - protein, 23 aa, Artificial Sequence
  Bacteria Phage T4 Fibritin
IKKIEEIEKKIEEIEKKIEEIEK

SEQ ID NO: 38 - protein, 26 aa, Artificial Sequence
  Bacteria Phage T4 Fibritin
YIPEAPRDGQAYVRKDGEWVLLSTFL

SEQ ID NO: 39 - protein, 78 aa, Artificial Sequence
  Chimeric N-Peptide SZN51
IEKKIEAIEKKIEAIEKKIEAIEKKIEQARQLLSGIVQQQNNLLRAIEAQQHLLQLTVWGIKQLQARILAVERYLKDQ

SEQ ID NO: 40 - protein, 82 aa, Artificial Sequence
  CC-Chimeric N-Peptide CC1ZN51
CCGGIKKEIEAIKKEQEAIKKKIEAIEKEIVQARQLLSGIVQQQNNLLRAIEAQQHLLQLTVWGIKQLQARILAVERYLKDQ

SEQ ID NO: 41 - protein, 7 aa, Artificial Sequence
  Linear Scaffold
XKXKXKX

SEQ ID NO: 42 - protein, 7 aa, Artificial Sequence
  Linear Scaffold
RKRKRKR

SEQ ID NO: 43 - protein, 7 aa, Artificial Sequence
  Linear Scaffold
EKEKEKE

SEQ ID NO: 44 - protein, 13 aa, Artificial Sequence
  Linear Scaffold
CHCCRKRKRKRNH

SEQ ID NO: 45 - protein, 8 aa, Artificial Sequence
  Chimeric N-Peptide CC1ZN36
CEKEKEKE

SEQ ID NO: 46 - protein, 66 aa, Artificial Sequence
  Chimeric N-Peptide CC1ZN36
CCGGIKKEIEAIKKEQEAIKKKIEAIEKEISGIVQQQNNLLRAIEAQQHLLQLTVWGIKQLQARIL

(19) United States
(12) Patent Application Publication
(10) Pub. No.: US 2015/0071954 A1
(43) Pub. Date: Mar. 12, 2015

(54) STABLE PEPTIDE MIMETICS OF THE HIV-1 GP41 PRE-HAIRPIN INTERMEDIATE

(71) Applicants: Joseph G. Joyce, Lansdale, PA (US); Chengwei Wu, Maple Glen, PA (US); Elizabeth A. Ottinger, Potomac, MD (US); Victor garsky, Blue Bell, PA (US)

(72) Inventors: Joseph G. Joyce, Lansdale, PA (US); Chengwei Wu, Maple Glen, PA (US); Elizabeth A. Ottinger, Potomac, MD (US); Victor garsky, Blue Bell, PA (US)

(21) Appl. No.: 14/386,514

(22) PCT Filed: Mar. 15, 2013
(86) PCT No.: PCT/US13/31831

§ 371 Date: Sep. 19, 2014

Related U.S. Application Data
(60) Provisional application No. 61/613,264, filed on Mar. 20, 2012.

(51) Int. Cl. C07K 14/005; A61K 39/21; C12N 7/00

(52) U.S. Cl. CPC C07K 14/005; C12N 7/00; A61K 39/21; C12N 2740/16122; C12N 2740/16134; A61K 2039/64
USPC 424/188.1; 530/395

(57) ABSTRACT

The present invention relates to a gp41 trivalent peptide mimetic having three gp41 N-peptides on a chemical scaffold which conformationally constrains the N-peptides into a trimeric coiled-coil to mimic gp41 presentation. The present invention also relates to N-peptides having the entire HIV gp41 NH2-terminal heptad repeat region and which are capable of forming gp41 peptide mimetics. Such peptide mimetics of HIV-1 gp41 pre-hairpin intermediates can be utilized in a vaccine for the treatment or prevention of HIV-1 infection through eliciting neutralizing antibodies.

### FIELD OF THE INVENTION

The present invention relates to conformationally constrained trivalent gp41 peptide mimetics and their use as immunogens to elicit neutralizing antibodies against HIV. The present invention also relates to N-peptides having the entire HIV gp41 NH₂-terminal heptad repeat region, or a modified version thereof, and which are capable of forming gp41 peptide mimetics.

### BACKGROUND OF THE INVENTION

Human Immunodeficiency Virus (HIV) is the etiological agent of acquired immune deficiency syndrome (AIDS) and related disorders. While effective treatments for AIDS are available, development of an efficacious prophylactic vaccine for the prevention of HIV-1 infection has been hampered by the inability to identify and optimize immunogens capable of inducing broadly neutralizing antibodies to prevent viral entry.

A considerable amount of research has been performed in evaluating use of the HIV-1 envelope glycoprotein as an immunogen. The HIV-1 envelope glycoprotein is synthesized as a 160 kDa precursor, which is cleaved by a host cell protease into a 120 kDa receptor-binding subunit (gp120) and a 41 kDa membrane-anchored subunit (gp41). Upon sequential binding of gp120 to CD4 and an associated CXCR4 or CCR5 chemokine co-receptor on permissible cells, extensive conformational changes take place in the envelope glycoprotein subunits such that the previously buried trimeric gp41 core is transiently exposed and ultimately results in fusion of the viral and cellular membranes and insertion of viral contents into the cytoplasm of the host cell.

Insertion of gp41 into the target host cell membrane is followed by an extensive rearrangement in which amphipathic C-terminal heptad repeat (CHR) regions of gp41 pack in an anti-parallel manner into hydrophobic pockets formed by the N-terminal heptad repeat (NHR) portion of the trimer to form a 6-helical “trimer of hairpins” structure. The trimer-of-hairpins structure is a bundle of six α-helices: three α-helices (formed by C-helix regions from three gp41 ectodomains) packed in an antiparallel manner against a central, three-stranded coiled-coil (formed by N-helix regions from three gp41 ectodomains). This rearrangement provides the requisite geometry and energy to bring the viral and cellular membranes into apposition which is followed by lipid mixing and membrane fusion.

Synthetic NHR and CHR peptides have been found to display potent antiviral activity and are believed to function through a dominant negative mechanism in which they bind to nascent fusion intermediates and block formation of the fusion-active 6-helix bundle. Soluble trimeric peptide mimetics of the pre-hairpin intermediate have been prepared by fusing heterologous trimerization domains derived from the yeast transcriptional factor GCN4 to varying residue lengths of the gp41 NHR. Such constructs are described in U.S. Pat. Nos. 7,960,504, and 7,811,577, and U.S. Patent Application Publication No. 20100092505. Similarly, recombinant 5-helix peptide, a single chain polypeptide consisting of alternating NHR and CHR sequences separated by short linkers produced by recombinant expression in bacteria, is described by U.S. Pat. Nos. 7,053,179 and 7,504,224. One approach used for stabilizing these peptides has been through the use of cysteine residues. See U.S. Pat. No. 7,811,577; Bianchi et al., 2009, Adv Exp Med Biol 611:121-3; Bianchi et al., 2010, Proc Natl Acad Sci USA 107:10655-10660; and Bianchi et al., 2005, Proc Natl Acad Sci USA 120:12903-12908. Other approaches for stabilizing gp41 peptides include those described in U.S. Pat. Nos. 7,728,106 and 7,604,804.

### SUMMARY OF THE INVENTION

The present invention relates to conformationally constrained trivalent gp41 peptide mimetics and their use as immunogens to elicit neutralizing antibodies against HIV. The gp41 peptide mimetics present all or a portion of the complete HIV-1 gp41 N-heptad repeat (NHR) region in a structurally stabilized form that mimics the native pre-hairpin fusion intermediate and which elicits an immune response capable of neutralizing HIV-1 virus.

Accordingly, the instant invention relates to a gp41 peptide mimetic comprising a scaffold core which is linked to three N-peptides wherein each N-peptide comprises an amino acid sequence comprising N36 (SGIVQQQNNLLRAIEAQQHLLQLTVWGIKQLQARIL; SEQ ID NO:1) or a modified version thereof, wherein the three N-peptides interact with each other to form a trimeric coiled-coil which mimics the pre-hairpin conformation of HIV gp41, with the proviso that the gp41 peptide mimetic is not (CCIZN36)₃. In particular embodiments, each of the three peptides is covalently linked to the scaffold core at a different point of attachment.

In certain embodiments, the gp41 peptide mimetic comprises a scaffold core which comprises tris(2-carboxyethyl)phosphine hydrochloride; tris-succinimidyl aminotriacetate; tris-(2-maleimidoethyl)amine; KTA-bromide or cholic acid. In particular embodiments, the scaffold core is KTA-bromide or cholic acid.

In other embodiments, the gp41 peptide mimetic comprises a scaffold core which is a linear polypeptide chain comprising three functionalized residues allowing attachment of three N-peptides. In particular embodiments, the scaffold core comprises:

(SEQ ID NO: 41)
a) CH₃CO-Ava-Lys-Ava-Lys-Ava-Lys-Ava-NH₂;
(SEQ ID NO: 42)
b) CH₃CO-Arg-Lys-Arg-Lys-Arg-Lys-Arg-NH₂;
(SEQ ID NO: 43)
c) CH₃CO-Glu-Lys-Glu-Lys-Glu-Lys-Glu-NH₂;
(SEQ ID NO: 44)
d) CH₃CO-Cys-Arg-Lys-Arg-Lys-Arg-Lys-Arg-NH₂;
or
(SEQ ID NO: 45)
e) CH₃CO-Cys-Glu-Lys-Glu-Lys-Glu-Lys-Glu-NH₂.

In yet other embodiments, the gp41 peptide mimetic comprises a scaffold core which is a carbocyclic scaffold comprising cyclohexane, cycloheptane or cyclooctane. In yet other embodiments, the gp41 peptide mimetic comprises a scaffold core which is a heterocyclic scaffold comprising pyrrolidine, oxolane, thiolane, piperidine, oxane, thiane, azepane, oxepane, thiepane, piperazine, morpholine, or thiomorpholine.

In certain embodiments of the invention, the N-peptide comprises N51

(SEQ ID NO: 4)
(QARQLLSGIVQQQNNLLRAIEAQQHLLQLTVWGIKQLQARILAVERYLK
DQ; N51-2B
(SEQ ID NO: 8)
(QIRELISKIVEQINNILRAIEAQQHALQLTVWGIKQLQARILAVERYLK
DQ
or
N51-3B
(SEQ ID NO: 9)
(QARQLLSGIVQQQNNLLRAIEAQQHALQLTVWGIKQLQARILAVERYLK
DQ.
In particular embodiments, the N-peptide consists
of N51
(SEQ ID NO: 4)
(QARQLLSGIVQQQNNLLRAIEAQQHLLQLTVWGIKQLQARILAVERYLK
DQ.

In certain embodiments, the N-peptides may comprise the same or different amino acid sequences. In a particular embodiment, the N-peptides consist of the same amino acid sequence.

In certain embodiments, one or more N-peptides are chimeric N-peptides which comprise:
a) a scaffold portion comprising a soluble α-helical region capable of forming a trimeric coiled-coil; and
b) a N-peptide portion comprising all or a portion of the HIV gp41 NH2-terminal heptad repeat region,
wherein the scaffold portion is fused in helical phase to the N-peptide portion, forming an α-helical domain, and wherein the three N-peptides interact with each other to form a trimeric coiled-coil. In certain aspects of this embodiment, the N-peptide portion of the chimeric N-peptide is fused in helical phase to the COOH-terminus of the scaffold portion of the chimeric N-peptide. In particular aspects of this embodiment, the scaffold portion of the chimeric N-peptide comprises:
a) The Suzuki-IZ coiled-coil motif (YGGIEKKIEAIEKKIEAIEKKIEAIEKKIEA (SEQ ID NO:31);
b) the IZ coiled-coil motif (IKKEIEAIKKEQEAIKKKIEAIEK (SEQ ID NO:34); or
c) the EZ coiled-coil motif (IEKKIEEIEKKIEEIEKKIEEIEK (SEQ ID NO:37).

The present invention also relates to a gp41 peptide mimetic which is:
a) KTA(N51)₃;
b) KTA(N51-2B)₃;
c) KTA(N51-3B)₃;
d) chA(N51)₃;
e) (CCIZN51)₃ or
f) SZN51.

In a particular embodiment, the gp41 peptide mimetic is KTA(N51)₃.

The present invention also relates to immunogenic compositions comprising the gp41 peptide mimetic of the present invention and a pharmaceutically acceptable carrier.

The present invention also relates to methods of eliciting an immune response in a mammalian host, comprising introducing into the mammalian host a prophylatically effective amount of the immunogenic compositions of the invention. In certain embodiments, the mammalian host is a human.

### BRIEF DESCRIPTION OF THE FIGURES

FIGS. 1A-B. Diagrammatic representation of HIV-1 gp41 peptide mimetics: A) Schematic structures in which the amphipathic coiled-coil helical HIV-1 N-peptides and SZ or IZ trimerization domains are depicted as cyclinders of different shading. The structures of various chemical scaffold cores is depicted; B) Chemical structures depicting trimerization strategies based on CCIZ, KTA, and cholic acid scaffolds and showing attachment of peptide sequences.

FIG. 2. Neutralizing antibody titers for individual animals in Guinea pig studies HIV-350 and HIV-365. Assay is p4/2R5 tested using viral strain V570A. Results are shown for pre-bleed (T=0) as open circles and for post-dose 3 bleed (T=11 weeks) as closed circles. Group geomean values are indicated by cross-bar. Assay Limit of Quantitation is shown by dotted line.

FIG. 3. ELISA responses by individual animal for NHP study HIV-360. Arrows indicate bleed dates following corresponding immunization with (CCIZN36)₃ at 0, 4, 8, and 34 weeks and homologous or heterologous antigen at 62 and 66 weeks. Curves are , (CCIZN36)₃ homologous; o, (CCIZN36)₃, /KTA(N51)₃ /5-helix; ▴, (CCIZN36)₃, /5-helix/KTA(N51)₃.

FIG. 4. DCBA responses by individual animal for NHP study HIV-360. Arrows indicate bleed dates following corresponding immunization with (CCIZN36)₃ at 0, 4, 8, and 34 weeks and homologous or heterologous antigen at 62 and 66 weeks. Curves are , (CCIZN36)₃ homologous; o, (CCIZN36)₃, /KTA(N51)₃ /5-helix; ▴, (CCIZN36)₃, /5-helix/KTA(N51)₃. Assay Limit of Quantitation is shown by dotted line.

FIGS. 5A-B. Neutralizing antibody titers for individual animals in NHP study HIV-360. Results are shown by neutralizing assay and virus tested for bleed collected two weeks post final immunization (T=68 weeks). Panel A, P4/2R5 assay using viruses V570A and HXB2. Panel B, A3R5 assay using viruses 9020.A13 and SC22.3C2. Group geomean values are indicated by solid cross-bar. Assay Limit of Quantitation is shown by dotted line. Brackets indicate significance between groups by Tukey test. To simplify axes labels, immunogens are abbreviated as “N36”, (CCIZN36)₃; “N51”, KTA(N51)₃; and “5H”, 5-helix.

FIG. 6. Neutralizing antibody breadth for individual animals in NHP study HIV-360. Results are shown by individual animal and group for bleed collected two weeks post final immunization (T=68 weeks). All results are from the A3R5 assay. Assay Limit of Quantitation is shown by dotted line. Virus clade and Tier designations are indicated in the legend. To simplify axes labels, immunogens are abbreviated as “N36”, (CCIZN36)₃; “N51”, KTA(N51)₃; and “5H”, 5-helix.

FIGS. 7A-B. Comparative neutralizing antibody titers for individual animals by study phase in NHP study HIV-360. Results are shown for the Phase 1 (panel A: homologous (CCIZN36)₃ dosing regimen) and Phase 2 (panel B: heterologous antigen administration) arms of the study by individual animal and group. All results are from the P4/2R5 assay. Group geomean values at either T=36 or T=68 weeks are indicated by cross-bars. Assay Limit of Quantitation is shown by dotted line. Panel A, neutralization results through end of Phase 1. T=0, pre-bleed; T=13, 5 weeks post-dose 3; T=36, 2 weeks post-dose 4; Panel B, neutralization results through end of Phase 2. T=62, bleed collected prior to initiation of homologous vs. heterologous comparison; T=68, 2 weeks post-final dose.

FIGS. 8A-B. Neutralizing antibody titers for individual animals determined by P4/2R5 and TZM-bl assays for bleed collected 2 weeks post-final immunization (T=38 weeks) in NHP study HIV-366. Results are shown for individual animal by group. All results are measured using virus V570_A_HXB2 virus in Panel A, P4/2R5 assay or Panel B, TZM-bl assay. Group geomean values are indicated by solid cross-bar. Assay Limit of Quantitation is shown by dotted line. Brackets indicate significance between groups by Tukey test. To simplify axes labels, immunogens are abbreviated as “N36”, (CCIZN36)₃; “N51”, KTA(N51)₃; and “5H”, 5-helix.

FIGS. 9A-B. Neutralizing antibody titers for individual animals determined by A3R5 assay. Panel A, Bleed at 2 weeks post final immunization (T=38 weeks) tested against virus Ce0393. Panel B, Bleed at 2 weeks post third dose immunization (T=26 weeks) tested against virus MW965. Group geomean values are indicated by solid cross-bar. Assay Limit of Quantitation is shown by dotted line. Brackets indicate significance between groups by Tukey test. To simplify axes labels, immunogens are abbreviated as “N36”, (CCIZN36)₃; “N51”, KTA(N51)₃; and “5H”, 5-helix.

### DETAILED DESCRIPTION OF THE INVENTION

In one aspect, the present invention relates to gp41 peptide mimetics comprising three gp41 peptides conformationally constrained into a trimeric coiled-coil by means of a scaffold. In particular, this aspect of the invention relates to gp41 peptide mimetics comprising a scaffold core which is linked to three N-peptides wherein each N-peptide comprises a N-peptide portion comprising N36 (SGIVQQQNNLLRAIEAQQHLLQLTVWGIKQLQARIL (SEQ ID NO:1) or a modified version thereof, wherein the three N-peptides interact with each other to form a trimeric coiled-coil which mimics the pre-hairpin conformation of HIV gp41.

In another aspect, the present invention relates to N-peptides having the entire HIV gp41 NH₂-terminal heptad repeat region, i.e., N51 (QARQLLSGIVQQQNNLLRAIEAQQHLLQLTVWGIKQLQARILAVERYLKDQ (SEQ ID NO:4)), or a modified version thereof, and which are capable of forming gp41 peptide mimetics, and the gp41 peptide mimetics formed therefrom.

The invention is based, in part, on Applicants' observation that, as illustrated in the Examples, in rodents, guinea pigs, and non-human primate immunogenicity studies, the conformationally constrained KTA(N51)₃ was superior over recombinant 5-helix peptide and (CCIZN36)₃ in eliciting neutralizing antibody responses against a panel of viral isolates.

While not wishing to be bound by any theory, it is believed that the present invention directs an immune response in vaccinated mammals to focus on the highly conserved gp41 pre-hairpin conformational intermediate which contains the specific D5-epitope neutralizing component. This is accomplished through conformationally constraining a gp41 peptide mimetic with a scaffold core to stabilize presentation of neutralizing epitopes in a trimeric coiled coil. In some embodiments, these stable, soluble, conformationally gp41 peptide mimetics potentially provide additional neutralizing epitopes distinct from the D5 epitope.

As used herein, “chimeric N-peptides” or “chimeric peptides” are defined as peptides which comprise all or a portion of the NH₂-terminal heptad repeat domain (NHR) of gp41 (generally at least N17 or N36), or a modified version thererof, fused to an α-helical scaffold protein capable of acquiring a trimeric coiled-coil conformation. The scaffold protein is a non-HIV sequence.

As used herein, the term “epitope” relates to a protein determinant capable of specific binding to an antibody. It is well known that epitopes usually consist of chemically active surface groupings of molecules such as amino acids or sugar side chains and usually have specific three dimensional structural characteristics, as well as specific charge characteristics. Conformational and nonconformational epitopes are distinguished in that the binding of the former but not the latter is lost in the presence of denaturing solvents.

As used herein, “heterologous” in reference to N-peptide constructs means N-peptide constructs which differs in (1) gp41 sequence (length and/or modifications), or (2) the identity or length of any scaffold domain (e.g., IZ, EZ, SZ).

As used herein, “HIV” is meant to represent HIV-1, HIV-2, or HIV-1 and/or HIV-2.

As used herein, “N-peptide” is defined as a peptide which comprises all or a portion of the NH₂-terminal heptad repeat domain (NHR) of gp41 (generally, at least N17 or N36), or a modified version thereof, and which is capable of acquiring a trimeric coiled-coil conformation whether alone or through the use of a scaffold domain.

As used herein, “neutralizing” is used as in the art, to denote the ability of an antibody to prevent, or reduce, viral infection in an in vitro cell/virus-based assay such as those described in Examples 1 and 2. Neutralizing activity may be measured quantitatively as the IC₅₀ value for that specific antibody. A “neutralizing antibody” or a “HIV neutralizing antibody” is shown in an art accepted infectivity assay to neutralize at least one HIV isolate.

As used herein, “scaffold core” relates to a chemical structure as disclosed and/or described herein which is a cyclical or linear chemical compound having at least three moieties each of which can be attached to a gp41 N-peptide, either directly or through a linker.

gp41 peptide mimetics of the invention mimic the internal, trimeric coiled-coil motif contained within the fusogenic conformation of an enveloped virus membrane-fusion protein, particularly the internal coiled-coil domain of the HIV gp41 ectodomain. These mimetics comprise three N-peptides, which may be chimeric N-peptides, together which form the trimeric coiled-coil characteristic of the gp41 pre-fusion intermediate. In some embodiments, the N-peptide are chimeric N-peptides which comprise a non-HIV, soluble, trimeric form of a coiled-coil fused in helical phase to all or a portion of the N-helix of HIV gp41, or a modified version thereof. In certain aspects of the invention, the three N-peptides are covalently-stabilized in a homotrimeric or heterotrimeric coiled-coil structure through the use of a scaffold core which conformationally constrains the N-peptides.

In certain embodiments, the N-peptides comprise all or a portion of the HIV gp41 NH₂-terminal heptad repeat domain, or a modified version thereof. The N-peptide may comprise, for example, N17, N36, N38, N44 or N51. In other embodiments, the N-peptides are chimeric peptides which comprise: 1) a scaffold portion comprising a soluble α-helical region capable of forming a trimeric coiled-coil; and 2) a N-peptide portion comprising all or a portion of the HIV gp41 NH₂-terminal heptad repeat region (for example, N17, N36 or N51), and optionally, 3) a cysteine portion comprising at least two cysteine residues, wherein the scaffold portion is fused in helical phase to the N-peptide portion, forming an α-helical domain and said cysteine portion, if present, is located outside of said α-helical domain at either the NH₂- or COOH-terminus. The presence of the cysteine portion assists in covalently constraining the chimeric peptides into a trimeric form. The covalent-stabilization of these N-peptides allows for the presentation of stable, exposed portions of the central, trimeric, N-helix coiled-coil of HIV gp41. The gp41 peptide mimetics comprise N-peptides which comprise all or a portion of the N-heptad repeat region derived from HIV-1 gp41. The HIV-1 gp41 ectodomain represents, approximately, 169 amino acid residues, residues 512-681 as numbered according to their position in the HIV-1 gp160 envelope protein of the reference strain HXB2. Within this ectodomain is a 4-3 heptad repeat region located adjacent to the NH₂-terminal portions of the ectodomain predicted to form α-helices. This N-heptad repeat is located, approximately, from amino acid positions 541-592 of gp160, respectively (see, e.g., Caffrey et al., 1998, EMBO J. 17:4572-4584).

The N-peptide domain of said peptide mimetics of the present invention comprises a sufficient amount of the N-helix region of gp41 to bind to the α-helices formed by the C-helix domain of the glycoprotein. Typically, 17 or more, or 36 or more, amino acid residues from the N-helix domain, up to and including all 51 of the residues of said domain, can comprise the HIV gp41 component of the N-peptides. Any sequence within the N-peptide region can be used so long as it presents an epitope. However, sequences shorter than about 36, 40, 45 or 50 amino acids may require additional peptides sequences that provide a scaffold domain, which will be discussed in greater detail below.

In one embodiment of the present invention, the N-peptides as described herein comprise at least the 36 amino acids located at the COOH-terminal half of the NHR of HIV-1 gp41, corresponding to residues 546 to 581 of the gp160 sequence (SGIVQQQNNLLRAIEAQQHLLQLTVWGIKQLQARIL (hereinafter referred to as “N36”; SEQ ID NO:1). In certain embodiments, N-peptides comprise, consist essentially of, or consist of, gp41 residues 546 to 583 (SGIVQQQNNLLRAIEAQQHLLQLTVWGIKQLQARILAG (hereafter referred to as “N38”; SEQ ID NO:2)). In certain embodiments, N-peptides comprise, consist essentially of, or consist of, gp41 residues 546 to 583 with six amino acids added at the NH₂-terminus (RGRGRGSGIVQQQNNLLRAIEAQQHLLQLTVWGIKQLQARILAG (hereafter referred to as “N44”; SEQ ID NO:3)). In certain embodiments, N-peptides comprise, consist essentially of, or consist of, gp41 residues 540 to 590 (QARQLLSGIVQQQNNLLRAIEAQQHLLQLTVWGIKQLQARILAVERYLKDQ (hereafter referred to as “N51”; SEQ ID NO:4)). Such peptide encompasses the HIV-1 gp41 N-heptad repeat region and a portion of the N-terminal polar domain. In these embodiments, the N-peptides were designed to allow presentation of the full length NHR in the context of a covalently stabilized conformationally constrained trimer. Additional N-peptides can include any sequences between N36 and N51. All numbering throughout the specification referring to the HIV gp160 sequence is based on HIV-1 isolate HXB2.

In other embodiments, the N-peptides may encompass additional HIV sequences derived from sequences either directly upstream or directly downstream of the NHR within the gp41 protein. For example, a chimeric peptide may encompass gp41 residues 528 to 590 (hereafter referred to as “N63”

(SEQ ID NO: 5)
STMGAASMTLTVQARQLLSGIVQQQNNLLRAIEAQQHLLQLTVWGIKQLQ
ARILAVERYLKDQ.

N-peptides can also be modified versions of the wild-type HIV N-helix heptad domain, provided that the resulting peptide is either an inhibitor of HIV infection of mammalian cells, as described herein, and/or capable of generating neutralizing antibodies targeting conformational epitopes of fusion intermediates. N-peptides with modifications made in the native NHR must maintain the trimerization ability and surface structure of the HIV N-peptide domain. For example, non-neutralizing, immunodominant regions (i.e., subunits of an antigenic determinant that are most easily recognized by the immune system and, thus, most influence the specificity of the induced antibody) may exist within the N-peptide sequence used to generate the N-peptides. Modified versions of the NHR (N36 or N51, as applicable) can have 1 amino acid change from the native gp160 sequence from HIV strain HXB2, up to two amino acid changes, up to three amino acid changes, up to four amino acid changes, up to five amino acid changes, up to six amino acid changes, up to seven amino acid changes or up to eight amino acid changes. Modifications can include amino acid inserts, deletions and/or substitutions. Generally, though, to maintain proper positioning, modifications are substitutions.

Alanine scanning of the NHR has identified an immunodominant region. This immunodominant region, which generates non-neutralizing antibodies, is located in the extreme COOH-terminal portion Amino acid residue arginine-579 (R579) appears to be critical for the binding of the non-neutralizing monoclonal antibodies; and residues glutamine-577 (Q577) and leucine-581 (L581) also participate in the binding but show variable contributions depending on the monoclonal tested. These residues that are involved in mouse monoclonal antibody binding form a ring at the bottom of the molecule that likely represents an immunodominant epitope in the NHR. Interestingly, the amino acid residues lining the hydrophobic pocket of the trimeric, N-helix coiled-coil are located further NH₂-terminal of this putative immunodominant epitope. The hydrophobic pocket has been identified as comprising a domain which binds to a newly identified, HIV-neutralizing antibody, D5 IgG; therefore, the hydrophobic pocket is thought to contain a putative neutralizing, conformational epitope (see U.S. Pat. No. 7,744,887). Thus, the N-peptide domain used to generate the N-peptides can be modified or shortened in an attempt to minimize the antigenic response of said identified, non-neutralizing immunodominant domain, focusing the immune response to the putative neutralizing epitope within the hydrophobic pocket. For example, in the modified versions of N36 or N51, the extreme COOH-terminal portion of N36 can be mutated at any one or more of the following residues: leucine-581 (L581), arginine-579 (R579), glutamine-577 (Q577) and/or glutamine-575 (Q575). It is preferable that each residue is mutated to an alanine (A) amino acid because alanine can participate in α-helix formation and, thus, will not disrupt the coiled-coil structure of the peptide. Additionally, alanine has a small side chain and, thus, will display the smallest possible binding surface for an antibody. Glycine or proline residues have no side chains and may be considered to be better choices for these mutations; however, said amino acids are known to disrupt α-helix conformation. In one embodiment of the present invention, the N-peptide comprises a sequence that is mutated at all four of the cited residues (L581A, R579A, Q577A and Q575A), forming an N-peptide domain designated as “N17A1a4” having the following sequence: LLQLTVWGIKALAAAIA (SEQ ID NO:6). The mutated amino acids are underlined.

The N-peptide portion of the N-peptides can also be modified to further stabilize the peptide as a whole. For example, the N-peptide domain can be modified to incorporate more stabilizing isoleucine residues into the sequence. Thus, for example, in one embodiment of the present invention, a N-peptide can be mutated at “a” and “d” packing positions to incorporate said isoleucine residues as follows: LIQLIWGIKQIQARIL (SEQ ID NO:7; designated “N17Ile”; mutated residues underlined).

Other modifications can be made in the N-peptide sequence in order to seek advantages in terms of trimer stabilization and/or presentation of the hydrophobic pocket and/or D5 epitope.

Modified versions of N51 sequence have been made to increase the solubility of the resulting trimer and/or reduce the propensity to aggregate. Examples of such peptides include N51-2B and N51-3B. N51-2B has the following sequence: QIRELISKIVEQINNILRAIEAQQHALQLTVWGIKQLQARILAVERYLKDQ (SEQ ID NO:8). N51-3B has the following sequence:

(SEQ ID NO: 9)
QARQLLSGIVQQQNNLLRAIEQQHALQLTVWGIKQLQARILAVERYLKDQ.

Other examples of N51 modified versions include peptides in Table 1.

TABLE 1
N51 modified versions

| | | |
|---|---|---|
| N51m1 | NIRQLLSGIVQQQNNLLRAIEAQQHL | 10 |
| | LQLTVWGIKQLQARILAVERYLKDQ | |
| N51m2 | QARQLISGIVQQQNNLLRAIEAQQHL | 11 |
| | LQLTVWGIKQLQARILAVERYLKDQ | |
| N51m3 | QIRQLISGIVQQQNNLLRAIEAQQHL | 12 |
| | LQLTVWGIKQLQARILAVERYLKDQ | |
| N51m4 | QARQLLSAIVQQQNNLLRAIEAQQHL | 13 |
| | LQLTVWGIKQLQARILAVERYLKDQ | |
| N51m5 | QIRQLISAIVQQQNNLLRAIEAQQHL | 14 |
| | LQLTVWGIKQLQARILAVERYLKDQ | |
| N51m6 | QARQLLSGIVQQINNLLRAIEAQQHL | 15 |
| | LQLTVWGIKQLQARILAVERYLKDQ | |
| N51m7 | NIRQLISAIVQQINNLLRAIEAQQHL | 16 |
| | LQLTVWGIKQLQARILAVERYLKDQ | |
| N51m8 | QARQLLSGIVQQQNNLLRAIWAQQHL | 17 |
| | LQLVVWGIKQLQARILAVERYLKDQ | |
| N51m9 | QARQLLSGIVQQQNNLLRAIEAQQHL | 18 |
| | LQLIVWGVKQLQAEILAVERYLKDQ | |
| N51m10 | QARQLLSGIVQQQNNLLRAIEAQQHL | 19 |
| | LQLTVWGIKQIQARILAVERYLKDQ | |
| N51m11 | QARQLLSGIVQQQNNLLRAIEAQQHL | 20 |
| | LQLTVWGIKQLQARILAIERYIKDQ | |
| N51m12 | QARQLLSGIVQQQNNLLRAIEAQQHL | 21 |
| | LQLTVWGIKQLQARILAVERYLKDI | |
| N51m13 | QARQLLSGIVQQQNNLLRAIEAQQHL | 22 |
| | LQLIVWGVKQIQARILAIERYIKDI | |
| N51m14 | QARQLLSGIVQQQNNLLRAIEAQQHL | 23 |
| | LQLVVWGNKQLQARVLAVERYLKDQ | |
| N51m15 | QIRQLISGIVQQINNILRAIEAQQHL | 24 |
| | LQLIVWGIKQIQARILAIERYIKDQ | |

It is understood that these same modifications as exemplified in the N51 sequences can be applied to shorter sequences as well.

Thus, specific modifications suitable for use for the compositions of the invention include substitutions at the following positions (all referring to the positions in HXB2): Q540, A541, Q543, L545, G547, Q550, Q552, L555, L565, T569, 1573, L576, I580, V583, L587 or Q590. Specific modifications include the following substitutions: Q540N, A541I, Q543E, L545I, G547A, G547K, Q550E, Q552I, L555I, L565A, L566I, T569V, T569I, I573V, I573N, Q575A, L576I, Q577A, R579A, I580V, L581A, V583I, L587I or Q590I.

The N-helix portion of HIV gp41 used to generate N-peptides can be isolated from HIV-1, HIV-2, another HIV strain or a strain from another lentiviral species (e.g., simian immunodeficiency virus (SIV), feline immunodeficiency virus (FIV) or Visna virus). The corresponding N-peptide sequences in similar HIV strains and/or immunodeficiency viruses of other species can be easily identified and are known in the art. Additionally, α-helical, coiled-coil domains have been identified in the membrane-fusion proteins of other enveloped viruses (see Singh et al., 1999, J Mol Biol. 290:1031-41).

Furthermore, for each of the peptide sequences described herein, the N-peptide domain can be extended by from one to twelve amino acids which are not part of the gp41 sequence. For example, Weissenhom et al. (1997, Nature 387:426-430) extends the N36 peptide by five amino acid residues at the NH₂-terminus and by seven amino acid residues at the COOH-terminus of the N36 peptide sequence: ARQLLSGIVQQQNNLLRAIEAQQHLLQLTVWGIKQLQARILAVERYLK (SEQ ID NO:25). Thus, the N-peptides may further comprise either all or a portion of seven additional amino acids, specifically AVERYLK (SEQ ID NO:26), located COOH-terminal of all or a COOH-terminal portion of the N36 peptide domain. Thus, in one embodiment, a N-peptide comprises a N-peptide domain designated as “N17+7”

(LLQLTVWGIKQLQARILAVERYLK (SEQ ID NO: 27)),
“N23 + 7”
(IEAQQHLLQLTVWGIKQLQARILAVERYLK (SEQ ID NO: 28)),
or
“N36 + 7”
(SGIVQQQNNLLRAIEAQQHLLQLTVWGIKQLQARILAVERYLK
(SEQ ID NO: 29)).

Additionally, the N-peptides may comprise all or a NH₂-terminal portion of the N36 peptide sequence plus up to an additional five amino acids located at the NH₂-terminus of said N-peptide, extending the N-peptide region further into the NH₂-terminal region. Thus, the N-peptides may further comprise either all or a portion of five amino acids located at the NH₂-terminus of the N36 peptide, specifically ASQLL (SEQ ID NO:30).

In certain embodiments a peptide-based scaffold domain may be required to maintain the conformation of the N-peptide region. The scaffold domain is non-HIV amino acid sequence so the resulting N-peptide is a chimeric N-peptide. The scaffold domain comprises a soluble α-helical domain capable of forming a trimeric coiled-coil and is fused in helical phase to the N-peptide, creating a continuous coiled-coil.

A coiled-coil is a protein structural motif consisting of two or more α-helices wrapped around each other with a superhelical twist. A simple pattern of amino acid residues determines the fold of a coiled-coil, consisting of a characteristic heptad repeat of amino acids designated by the letters “a” through “g”. It has been determined that the first and fourth positions of the heptad repeat, the “a” and “d” positions, respectively, form the interior of the interacting strands of the coiled-coil and are generally hydrophobic. The scaffold domain contained within the chimeric N-peptide forms trimeric coiled-coil structures within the gp41 peptide mimetics of the invention so as to mimic the internal, trimeric coiled-coil present in the pre-hairpin and trimer-of-hairpins structures formed by N-helices of three gp41 ectodomains.

In one embodiment of the present invention, the scaffold domain is fused to the NH₂-terminus of the N-peptide region. In another embodiment, the scaffold domain is fused to the COOH-terminus of the N-peptide region. In a still further embodiment, the scaffold domain can be divided such that portions of said domain are located at both the NH₂- and COOH-termini of the N-peptide region.

One can use any coiled coil motif known to trimerize to stabilize the gp41 trimers. Coiled-coil motifs can be selected from a variety of sources. The scaffold domains within the chimeric N-peptides described herein particularly include the isoleucine zipper motif disclosed in Suzuki et al. (1998, Protein Eng. 11: 1051-1055; hereinafter “Suzuki-IZ”) and the GCN4-pI_{Q}I coiled-coil motif disclosed in Eckert et al. (1998, J. Mol. Biol. 284:859-865 and International Patent Application Publication No. WO02/024735), and truncated and/or modified versions thereof.

The Suzuki-IZ coiled-coil motif has the following amino acid sequence: YGGIEKKIEAIEKKIEAIEKKIEAIEKKIEA (SEQ ID NO:31). The “a” positions of the heptad repeat that comprise the Suzuki-IZ motif ([(IEKKIEA)ₙ; (SEQ ID NO:32)]ₙ) are underlined.

The GCN4-pI_{Q}I coiled-coil motif has the following amino acid sequence: RMKQIEDKIEEILSKQYHIENEIARIKKLIGER (SEQ ID NO:33). The “a” positions of this helical motif are also underlined.

The IZ domain (IKKEIEAIKKEOEAIKKKIEAIEK (SEQ ID NO:34)) is a modified isoleucine zipper based on a design described by Suzuki et al. (1998, Protein Eng. 11: 1051-1055) that is helical and trimeric in solution.

The Suzuki-IZ, GCN4-pI_{Q}I, and other scaffold domains can be changed by the addition, substitution, modification and/or deletion of one or more amino acid residues. “Suzuki-IZ-like” and “GCN4-pI_{Q}I-like” scaffold domains are defined herein as coiled-coil motifs that comprise either a portion of the “Suzuki-IZ” or “GCN4-pI_{Q}I” coiled-coils, respectively, or a modified version of all or a portion of said respective coiled-coils. The Suzuki-IZ-like and GCN4-pI_{Q}I-like scaffold domains must consist of a sufficient portion (i.e., a sufficient length) of the Suzuki-IZ and GCN4-pI_{Q}I trimeric coiled-coil domains, respectively, or modified versions thereof, such that they form soluble, trimeric (helical) coiled-coils. The tolerance for changes in the amino acid sequence of the scaffold protein will depend on whether the changed amino acids serve structural and/or functional roles. Thus, mutated or modified scaffold proteins used herein must retain the ability to form trimeric coiled-coils. Additionally, the gp41 peptide mimetics of the invention comprised of three N-peptides, at least one of which is generated with a mutated/modified scaffold domain, must retain either the ability to inhibit HIV infectivity with potencies in, for example, at least the low nanomolar concentration range, e.g., 1-5 nM, or the capacity to bind gp41-specific antibodies that recognize conformational epitopes located in the N-helix coiled-coil. Modification of the scaffold protein may provide several advantages. For example, the outside surface of the chimeric peptides of the present invention can be varied to enhance bioavailability (e.g., increase solubility of the peptide), decrease toxicity and avoid immune clearance. The availability of multiple versions of the chimeric peptides of the present invention encompassing alternative scaffolds would help to circumvent this problem by evading preexisting antibodies. The scaffold protein may also be modified in an attempt to make the scaffold domain of the chimeric peptide less immunogenic, for example, by introducing glycosylation or pegylation sites on its external surface. Furthermore, the scaffold domain may be modified to facilitate the conjugation of said peptide mimetic to an immunogenic carrier or an affinity resin.

The IZ scaffold motif, described above, represents a portion of the Suzuki-IZ coiled-coil motif that has been significantly altered in the “e” and “g” positions and possesses an isoleucine to glutamine (I→*Q) substitution at an “a” position (see International Patent Application Publication No. WO02/024735). The amino acid sequence of the “IZ” scaffold domain is IKKEIEAIKKEQEAIKKKIEAIEK (SEQ ID NO:34; “a” positions are underlined), wherein the NH₂-terminus is acetylated and the COOH-terminus amidated.

Shortened versions of the IZ scaffold domain can also be generated for incorporation into peptide mimetics. A specific example of a shortened IZ-like domain represents 17 amino acids of the IZ scaffold: IKKEIEAIKKEQEAIKK (SEQ ID NO:35; designated as “IZ17”; “a” positions are underlined).

In order to maintain proper helical structure when generating alternative peptide mimetics having longer HIV sequence segments, this “IZ” scaffold may need to be extended by from one to a few amino acids, generating “IZ-like” scaffold domains. For example, IZN23 and IZN36 are chimeric N-peptides also disclosed in Eckert et al., 2001, Proc Natl Acad Sci USA 98:11187-11192. The amino acid sequence of IZN36 is IKKE IEAIKKE QEAIKKK IEAIEKE ISGIVQQ QNNLLRA IEAQQHL LQLTVWG IKQLQAR IL (SEQ ID NO:36), respectively. The “a” positions of the peptides are underlined. For example, the IZ-like scaffold domains of IZN36 can be extended by one or, preferably, two amino acids. This may be required to maintain proper “a” through “g” spacing and, thus, facilitates generation of an α-helical conformation. The amino acids chosen to extend the scaffold domain in this manner should enable electrostatic interaction between adjacent helices (see Suzuki et al., 1998, Protein Eng. 11: 1051-1055). When generating chimeric N-peptides to be stabilized, one of skill in the art will appreciate that the scaffold domain may need to be minimally altered, as seen with IZN36, in order to maintain the helical conformation of the resulting peptide. Similar changes can be made with other scaffolds.

In another embodiment, the scaffold domain comprises a modified Suzuki-IZ-like domain, designated as the “EZ” scaffold, having the following amino acid sequence: IKK IEEIEKK IEEIEKK IEEIEK (SEQ ID NO:37; “a” positions are underlined).

In another embodiment, the 26 amino acid trimeric motif of the bacteriophage T4 fibritin trimeric (FT) sequence, YIPEAPRDGQAYVRKDGEWVLLSTFL (SEQ ID NO: 38), is used as a scaffold domain. Similar motifs based on fibritin are known in the art.

One example of a preferred chimeric N-peptide is SZN51

(SEQ ID NO: 39)
IEKKIEAIEKKIEAIEKKIEAIEKKIEQARQLLSGIVQQQNNLLRAIEAQ
QHLLQLTVWGIKQLQARILAVERYLKDQ.

As described above, the amino acid sequence of the scaffold domain of the peptide mimetics can be modified and/or shortened; however, in doing so, the resulting chimeric peptides must retain the ability to form a trimeric coiled-coil representing a stable, faithful mimetic of the internal, N-helix coiled-coil of gp41.

In certain embodiments, the chimeric N-peptides are covalently stabilized. A peptide mimetic may comprise a N-peptide fused in helical phase to a scaffold domain, wherein the peptide sequence optionally further comprises at least two cysteine residues located at either the NH₂- or COOH-terminus and, preferably, outside of the core helical region of the chimeric peptide. These peptides are referred to as CC-chimeric N-peptides. In such an embodiment, three, identical or substantially similar, cysteine-containing chimeric peptides are then covalently-stabilized in a homotrimeric or heterotrimeric molecule via intermolecular disulfide bonds formed under oxidizing conditions between juxtaposed cysteine residues on closely associated chimeric peptide chains. The covalently-stabilized, homotrimeric or heterotrimeric coiled-coil is formed either by exposing a pre-formed, trimeric coiled-coil to an oxidizing environment or by promoting the association of individual peptide chains into a coiled-coil conformation under oxidizing conditions. The added cysteine residues are located outside of the α-helical domain of the chimeric peptide, ensuring high conformational freedom, and optionally separated from the core, chimeric peptide sequence by a linker or spacer region. See U.S. Pat. No. 7,811,577.

The cysteine residues present in CC-chimeric N-peptides described herein are consecutive amino acid residues. Therefore, a CC-chimeric N-peptides described herein can comprise at least two, consecutive cysteine residues-located at either the NH₂- or COOH-terminal ends of the peptide, outside of the core α-helical domain of the chimeric peptides, and optionally, separated from the core α-helical domain by a space/linker region. CC-chimeric N-peptides described herein can also comprise exactly two, consecutive cysteine residues at either the NH₂- or COOH-terminal ends of the peptide, outside of the core α-helical domain of the chimeric peptides, and optionally, separated from the core α-helical domain by a space/linker region. One skilled in the art can also envision that the cysteine residues described herein do not necessarily have to be consecutive residues, and thus, it may be possible to include a minimal number of amino acid residues between said cysteine residues. It is important, however, that the cysteine residues are not spaced sufficiently far apart so as to enable the generation of intramolecular disulfide bonds between cysteine residues on the same polypeptide chain, leaving them incapable of forming intermolecular disulfide bonds between the individual CC-chimeric N-peptides in a trimeric, coiled-coil conformation.

The two, consecutive cysteine residues participate in disulfide bond linkages with juxtaposed cysteine residues on closely associated CC-chimeric N-peptides that are formed upon oxidation of the peptides. In embodiments, where two, consecutive glycine residues are present, the glycine residues represent a spacer region, separating the cysteine residues from the α-helical domain of the core chimeric peptide sequence. The glycine spacer region ensures that the cysteine residues are not embroiled in the helical secondary structure of the core peptide sequence, helping to free said cysteines to participate in disulfide linkages. Covalent cross-links between individual proteins (i.e., intermolecular) or within a single polypeptide chain (i.e., intramolecular) can be formed by the oxidation of cysteine residues. Disulfide bonds are formed by the oxidation of the thiol (—SH) groups in cysteine residues. Intramolecular disulfide bonds stabilize the tertiary structures of proteins, while those that occur intermolecularly are involved in stabilizing protein structure involving one or more polypeptides. Covalent cross-links between individual peptides/proteins can also be formed by chemoselective reactions (e.g., formation of thioether bonds) imposed by incorporating unique, mutually reactive groups into said peptides/proteins to be covalently-linked—one within each segment to be joined (reviewed in Lemieux G. A. and Bertozzi C. R., 1998, Trends Biotechnol. 16:506-513; and Borgia, J. A. and Fields G. B., 2000, Trends Biotechnol. 15:243-251). The cysteine residues that are added to the core α-helical domain of chimeric peptides create disulfide bonds upon oxidation, covalently-stabilizing the trimeric structure formed by three, identical CC-chimeric N-peptides.

The cysteine residues described herein may be added to the NH₂-terminus or COOH-terminus of the core chimeric N-peptide to generate CC-chimeric N-peptides. For example, two cysteine residues can be engineered to occupy the first two amino acid residues at the NH₂-terminus of a CC-chimeric N-peptide, wherein the scaffold coiled-coil domain is located in the NH₂-terminal half of the chimeric peptide. This arrangement ensures that the two engineered cysteine residues are least likely to interfere with the α-helical structure of the N-peptide portion of the chimeric peptide and/or the functionality of said HIV domain, e.g., to interact with C-helices. Alternatively, two cysteine amino acid residues can be engineered to occupy the last two amino acid residues at the COOH-terminus of a CC-chimeric N-peptide, wherein the scaffold; coiled-coil domain is located in the NH₂-terminal half of the chimeric peptide. This may be necessary, for example, if there is difficulty conjugating a CC-chimeric N-peptide to an immunogenic carrier or an affinity resin via the non-HIV scaffold portion of the chimeric peptide due to the presence of the Cys-Cys sequence located adjacent to the scaffold domain. Two cysteine residues can also be engineered to occupy the first two amino acid residues at the NH₂-terminus of a CC-chimeric N-peptide, wherein the N-peptide domain is located in the NH₂-terminal half of the chimeric peptide. Two cysteine residues can be engineered to occupy the last two amino acid residues at the COOH-terminus of a CC-chimeric N-peptide, wherein the N-peptide domain is located in the NH₂-terminal half of the chimeric peptide. Switching the orientation of the N-peptide and scaffold domains may impact the ability of the resulting CC-chimeric N-peptide to inhibit viral-host cell membrane fusion.

A preferred CC-chimeric N-peptide is CCIZN51:

(SEQ ID NO: 40)
CCGGIKKEIEAIKKEQEAIKKKIEAIEKEIVQARQLLSGIVQQQNNLLRA
IEAQQHLLQLTVWGIKQLQARILAVERYLKDQ.

In alternative embodiments, stabilization occurs through incorporation of an electrophilic moiety to either terminus of the core chimeric peptides for participation in stabilizing disulfide and/or thioether bonds, respectively, between said peptides. Said electrophilic moieties are optionally separated from the α-helical domain of the chimeric peptides by a linker or spacer region. Thus, said structure can be attained by the trimerization and covalent-stabilization of a single CC-chimeric N-peptide with two derivatized-chimeric N-peptides each having an electrophilic moiety, wherein a thioether bond is formed between each thiol-reactive functional group present in the engineered cysteine residues of the CC-chimeric N-peptide and the electrophilic moiety (e.g., an alkyl halide moiety or a Michael acceptor) of each derivatized-chimeric N-peptide. The disulfide or chemoselective covalent bond linkages between the chimeric peptides ensure that peptide monomers (i.e., single, chimeric peptide subunits of the homotrimeric or heterotrimeric coiled-coil structure) do not dissociate, even at very low concentrations.

In certain embodiments, stabilization occurs through incorporation of functionalities able to mediate “click” chemistry couplings to either terminus of the core chimeric peptides for participation in stabilizing covalent bonds between said peptides. Said “click” moieties are optionally separated from the α-helical domain of the chimeric peptides by a linker or spacer region. In such an embodiment, the stabilized trimer may be formed by reaction of a single XX-chimeric N-peptide with two Y-derivatized-chimeric N-peptides, wherein “X” and “Y” represent cognate moities of the “click” reaction pair (e.g., the Huisgen 1,3 dipolar cycloaddition in which “X” constitutes an alkyne moiety and “Y” constitutes an azide moiety) of each derivatized-chimeric N-peptide. The chemoselective covalent bond linkages between the chimeric peptides ensure that peptide monomers (i.e., single, chimeric peptide subunits of the homotrimeric or heterotrimeric coiled-coil structure) do not dissociate, even at very low concentrations.

An alternative strategy used by Louis et al. (2001, J. Biol. Chem. 276:29485-29489) to generate an internal, trimeric coiled-coil of the gp41 ectodomain mutated actual residues within the N-helix domain to cysteine residues to stabilize by intermolecular disulfide bridges. The disulfide bonds are generated between cysteine residues that are incorporated into the six-helix bundle by mutating residues 576-578 of the gp41 ectodomain, located within the N-helix region, to cysteine-cysteine-glycine (Cys-Cys-Gly). One of said mutated amino acid residues was located in the “d” position of the α-helical domain, known to be one of two positions of the heptad repeat that forms the interior of the interacting strands of the coiled-coil and also highly conserved among HIV-1 clades (Dong et al., 2001, Immunol. Lett. 75:215-220).

An alternative method of stabilizing a CC-chimeric N-peptide described herein is to add the cysteine residues to the opposite terminus of the peptide. Thus, initially, if a trimeric coiled-coil formed with CC-chimeric N-peptides stabilized via disulfide bonds between cysteine residues residing at one terminus of said peptides does not display either an ability to inhibit HIV infectivity with a high potency or the capacity to bind an antibody that recognizes a conformational epitope located in the N-helix domain, one of skill in the art may generate a similar covalently-stabilized trimeric coiled-coil having the stabilizing cysteine residues located at the opposite terminus of the CC-chimeric N-peptides.

When stabilizing a trimeric coiled-coil of the present invention via any of the methods described above, it is important that the moiety incorporated within the two, derivatized-chimeric N-peptides is located at the same terminus of said peptides. It can then be determined if the location of the stabilizing mechanism affects the functionality of the covalently-stabilized chimeric peptides. Moving the stabilizing mechanism to the opposite terminus may have a further stabilizing affect if the end to which the unit is added is less stable than the opposite end of the peptide. Often, the N-peptide portion of the chimeric N-peptides described herein is less stable than the scaffold portion of the peptide. Thus, moving the stabilizing unit from the scaffold terminus of the peptide to the N-peptide terminus may increase the stability of the resulting trimeric coiled-coil.

The three-dimensional structure of a D5 epitope is mimicked and stabilized by restricting the conformation of the trimeric coiled-coil formed by 3 N-peptides as described herein. In an alternative strategy to the CCIZ approach, a scaffold core is employed for coupling and positioning of N-peptides so as to position them in a manner that they are free to self-associate with comcommitant formation of the trimeric coiled-coil. Therefore, in certain embodiments, the scaffold core does not comprise two adjacent cysteine residues (i.e., CC). The scaffold core contains at a minimum three positions for covalent attachment of the N-peptides. Said attachment positions are maintained at an optimal spacing and distance from other structural elements of the scaffold core to facilitate self-association of the N-peptides to form a trimeric coiled-coil. A scaffold core is as a linear or cyclic compound comprising three or more reactive groups whereby a peptide can be covalently attached. Thus, as used herein, a multivalent or more specifically a trivalent, peptide is a compound comprising three peptides covalently attached to a scaffold core. The general structure of a scaffolded trivalent peptide is represented as:

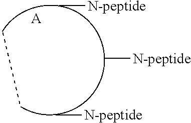

A specific example of a scaffolded peptide would be any N-peptide containing a cysteine residue that is able to react with a bromide or maleimide moiety present at one or more attachment points on a scaffold with subsequent formation of a covalent thioether bond.

The locations of at least three linkages are chosen such that the resulting conformation of the D5 epitope in the gp41 peptide mimetic resembles the native conformation of said epitope in gp41. The gp41 peptide mimetics of the invention are influenced by the type of scaffold that is used, i.e., its structure, since the size and the shape of the scaffold will influence the overall structure of the peptide mimetic. Based on the guidance provided herein, a skilled person in the art is well capable of designing a peptide mimetic of the invention with a conformation closely resembling the native conformation of the D5 epitope of gp41. The point of attachment of scaffold to N-peptide is preferably not located within the D5 epitope, because such linkage would disturb the conformation and/or accessibility of the epitope. It is for instance possible to produce several compounds with linkages at different locations and to experimentally determine the ability of said compounds to bind D5 and/or to inhibit D5 binding in a competitive binding assay (e.g., DCBA) as a measure of proper epitope presentation. Similarly, presentation of the NHR in an appropriate coiled-coil conformation may be assessed by the potency of said compounds in a viral entry inhibition-based assay.

A compound with optimal presentation of the NHR conformation and/or D5 epitope is preferably selected. It is also possible to produce several compounds with different kinds of scaffolds, either linked at identical or different locations of an amino acid sequence, and to experimentally determine the presentation of the NHR conformation and/or D5 epitope of the resulting compounds.

Thus, the N-peptides are attached to a scaffold, either directly or indirectly, via a linker, and by the formation of at least one bond within said amino acid sequence.

Suitable molecular scaffold cores include mono- and poly-carbocyclic compounds with individual ring structures up to 10 carbons or heterocyclic compounds having at least one atom other than carbon in the ring structure, most commonly nitrogen, oxygen or sulfur. Examples include cyclobutane, cyclopentane, cyclohexane, cyclooctane, pyran, pyrrolidine, oxolane, thiolane, piperidine, oxane, thiane, azepane, oxepane, thiepane, piperazine, morpholine, thiomorpholine, and derivatives thereof.

One example of a carbocyclic chemical scaffold is cis,cis-1,3,5-trimethyl cyclohexane-1,3,5-tricarboxylic acid (Kemp's acid) in which thiol-reactive bromoacetyl groups are introduced following derivatization of the carboxylic acid functionalities with diaminoethane as described in Xu, et al (Xu, W. et al., 2007 Chem Biol Drug Des 70: 319-328). Kemp's acid presents a favored chair conformation in which the three carboxyls on the cyclohexane ring occupy axial positions and thus provide a favored triaxial orientation to assemble N-peptides.

In another embodiment, the N-peptides are coupled to a scaffold that is based on or consists of multiple fused ring structures. Two carbocyclic or heterocyclic rings that share a carbon-carbon bond are said to be fused. Suitable scaffolds may include fused ring derivatives of any of the preceeding carbocyclic or heterocyclic compounds herein described. Specific examples include cholesterol, cholic acid, and derivatives thereof and terphenyls as disclosed in U.S. Pat. No. 7,312,246.

Other examples of chemical scaffolds include carbohydrate-based, and scaffolded maleimide clusters as descrbed in U.S. Pat. No. 7,524,821 which are useful in multivalent peptide and protein assembly. Such maleimide clusters take advantage of the well-established, highly efficient Michael addition of a thiol group to an electrophilic moiety (Kitagawa et al., 1976, J. Biochem. (Tokyo). 79:233-6; Peeters et al., 1989, J. Immunol. Methods. 120:133-43). Thus, the topology of the multivalent peptides can be controlled by defined spatial orientation of the maleimide functionalities on the rigid scaffold core. Alternative thiol reactive compounds which can be substituted for maleimide are iodoacetic acid, bromoacetic acid, iodoacetamide and pyridyl disulfide. The disulfide linkages formed with pyridyl disulfide are cleavable by methods well known in the art.

A preferred embodiment of the instant invention is a maleimide cluster comprising a core molecule wherein three or more maleimides are each attached to the core. Another preferred embodiment of the invention is a maleimide cluster comprising a carbohydrate core wherein three maleimides are each attached to the core. Still another preferred embodiment of the invention is a maleimide cluster comprising a carbohydrate core wherein three, four, five or six maleimides are each attached to the core by a linker.

A preferred embodiment of the invention is a maleimide cluster comprising a cholic acid core wherein three, four, five or more maleimides are each attached to the core. Another preferred embodiment of the invention is a maleimide cluster comprising a cholic acid core wherein three, four, five or more maleimides are each attached to the core by a linker.

A preferred embodiment of the invention is a maleimide cluster comprising cyclodextrin wherein three or more maleimides are each attached to the cyclodextrin by a linker. A preferred embodiment of the invention is a maleimide cluster comprising at least two cores wherein each core contains one or more maleimides. Another preferred embodiment of the invention is a maleimide cluster comprising a polyol core, wherein three or more maleimides are each attached to the core. A further preferred embodiment of the invention is a maleimide cluster comprising a polyol core, wherein three or more maleimides are each attached to the core by a linker.

Scaffolds can also be monosaccharides, polyols and oligosaccharides. Monosaccharides that can serve as a scaffold of the instant invention include but are not limited to dihydroxyacetone, R and L enantiomeric and anomeric forms of glyceraldehyde, threose, erythrose, erythrulose, ribose, arabinose, xylose, lyxose, ribulose, xylulolse, allose, altrose, glucose, mannose, gulose, idose, galactose, talose, psicose, fructose, sorbose and tagatose. Polyols or polyalchohols that can serve as a scaffold compound include, but are not limited to, glyceritol, threitol, erythritol, ribitol, arabinitol, xylitol, lyxitol, allitol, altritol, glucitol, mannitol, galactitol, talitol, gulitol, iditol, sorbitol, mannitol, glycerol, inositol, maltitol, lactitol, dulcitol and adonitol. Oligosaccharides that can serve as a scaffold compound include, but are not limited to, disaccharides comprising any combination of monosaccharides described above and cyclic oligosaccharides comprising the monosaccharides described above. Cyclodextrin and cyclofructin are examples of cyclic oligosaccharides that can be used in the scaffold of the instant invention. Cyclodextrins are cyclic (α-1,4)-linked oligosaccharides and include, but are not limited to 5-13 α-D-gluco-pyranose, cyclomannin, cycloaltrin and cyclogalactin. Cyclodextrins comprise a hydrophobic core, capable of carrying compounds. A maleimide cluster may further comprise several linked core compounds comprising reactive maleimide moieties. A chemical scaffold core can also be based on cholic acid, cholesterol, cyclic peptides, porphyrins and calyx [4] arene, carbohydrates and polyamines Specific polyamines should be triamines, for example, diethylene triamine penta-acetic acid, pentamethyldiethylene triamine, tris-2 aminoethyl amine, dipropylenetriamine, and the like.

A scaffold may also be a non-cyclic or linear molecule which contains a minimum of three functional groups that can serve as points of attachment for N-peptides. Examples of this class of scaffold include but are not limited to tris(2-carboxyethyl)phosphine hydrochloride; tris-succinimidyl aminotriacetate; tris-(2-maleimidoethyl)amine; TRIS(Boc-β-Ala-TRIS-(OH)₃; and TREN (Tris(2-aminoethyl)amine) TRIS scaffold is described in Cai et al., 2007, Bioorganic Chemistry 35:327-337. TREN (Tris(2-aminoethyl)amine) is described in Kwak et al., 2002, J. Am. Chem. Soc. 124:14085-14091. Additional structures suitable as scaffold cores can be found in U.S. Pat. No. 7,604,804, which is incorporated by reference in its entirety.

In another embodiment of the invention, the N-peptides are coupled to a linear scaffold that is based on or which comprises amino acids containing side chains capable of being derivatized for attachment to an activated N-peptide. Said amino acid residues include Lys, Arg, His, Glu, Asp, Cys, Sec, and derivatives thereof. Said amino acid residues may be part of a polypeptide chain that contains at least three reactive groups in which spacing between the reactive groups is suitable to optimally restrict the conformation of the N-peptides forming the trimeric coiled-coil.

One example of a linear scaffold comprises a peptide Ac-X-Lys-X-Lys-X-Lys-X-NH₂ in which three homologous or heterologous N-peptides can be attached to the β-amino side chain of the lysine residues (RNH₂), and in which X can be any amino acid that provides sufficient spacing for proper orientation of the N-peptides or modifies charge, hydrophobicity, or other physical parameters of the scaffold. In a preferred embodiment of the instant invention, X is 5-aminopentanoic acid. In another preferred embodiment, X is Arg, or Glu.

Specific examples of peptides constrained by a linear scaffold include

Ac-Ava-Lys(N44)-Ava-Lys(N44)-Ava-Lys(N44)-Ava-NH₂
Ac-Ava-Lys(N38)-Ava-Lys(N38)-Ava-Lys(N38)-Ava-NH₂
Ac-Cys-Arg-Lys(N38)-Arg-Lys(N38)-Arg-Lys(N38)-Arg-
NH₂
Ac-Cys-Glu-Lys(N38)-Glu-Lys(N38)-Glu-Lys(N38)-Glu-
NH₂

where Ava represents δ-amino valeric acid.

Examples of the linear scaffolds include:

SEQ ID NO: 41
CH₃CO-Ava-Lys-Ava-Lys-Ava-Lys-Ava-NH₂;
SEQ ID NO: 42
CH₃CO-Arg-Lys-Arg-Lys-Arg-Lys-Arg-NH₂;
SEQ ID NO: 43
CH₃CO-Glu-Lys-Glu-Lys-Glu-Lys-Glu-NH₂;
SEQ ID NO: 44
CH₃CO-Cys-Arg-Lys-Arg-Lys-Arg-Lys-Arg-NH₂;
or
SEQ ID NO: 45
CH₃CO-Cys-Glu-Lys-Glu-Lys-Glu-Lys-Glu-NH₂;.

It will be recognized by one skilled in the art that a variety of chemistries may be used to covalently attach the N-peptide to a reactive functionality on the scaffold core. In one preferred embodiment, this attachment comprises a thioether bond between a thiol group on the N-peptide and an electrophilic moiety on the scaffold because said bond is readily formed in aqueous solution at neutral or slightly basic pH and because the thiol functionality on the N-peptide may be provided as a cysteine residue or as a thiol derivative on either terminus of the peptide. The location of a thioether bond internal to an amino acid sequence can easily be regulated by regulating the location of free cysteine residues. In a particularly preferred embodiment a thioacetyl functionality, which serves as a precursor of thiol functionality, is located at the N-terminus of the first or the C-terminus of the last amino acid position of the amino acid sequence, in order to optimally restrict the conformation of the amino acid sequence.

Other kinds of bonds are also suitable for restricting the conformation of an immunogenic compound of the invention. For instance, a disulfide bond (also called an SS-bridge) may be selectively formed between free cysteine residues without the need to protect other amino acid side chains. Furthermore, disulfide bonds are easily formed by incubating in a basic environment. Preferably a disulphide bond is formed between two cysteine residues, since their sulfhydryl groups are readily available for binding. The location of an SS-bridge within an amino acid sequence is easily regulated by regulating the location of free cysteine residues. In a particularly preferred embodiment said cysteines are located around the first and last amino acid position of the amino acid sequence, in order to optimally restrict the conformation of the amino acid sequence.

In another embodiment, Se—Se diselenium bonds can be used. An advantage of diselenium bonds is the fact that these bonds are reduction insensitive. Hence, peptide mimetics comprising a diselenium bond are better capable of maintaining their conformation under reducing circumstances, for instance present within an animal body. Furthermore, a diselenium bond is preferred when a free SH-group is present within the peptide mimetic, which SH-group is for instance used for a subsequent coupling reaction to a carrier. Such free SH-group is not capable of reacting with a diselenium bond.

In another embodiment, a metathesis reaction is used in order to form said bond. In a metathesis reaction two terminal CC-double bonds or triple bonds are connected by means of a metal-catalysed rearrangement reaction. Acceptable catalysts are Schrock molybdenum(VI) or tungsten(VI) alkylidenes or the Grubbs ruthenium carbenoids. The terminal CC-double or CC-triple bonds required for this reaction are introduced into a peptide either via alkylation of the peptide NH-groups, for instance with allyl bromide or propargyl bromide, or via incorporating a non-natural amino acid with an alkenyl- or alkynyl-containing side chain into the peptide.

In a preferred embodiment the bond between N-peptide and scaffold core is formed using bromine-thiol coupling. For instance, a bromoacetyl moiety on the scaffold core is coupled to a sulfhydryl moiety of a free cysteine which is preferably present at the N-terminus of the peptide. Alternatively, a thiol moiety on the scaffold core may be coupled to a bromoacetyl moiety introduced on the NH₂ terminus of the peptide or on the ε-amino side chain of a lysine residue (RNH₂) which is preferably present at the N-terminus of the peptide.

In a further embodiment a CO₂H-side chain of an aspartate or glutamate residue is coupled to an amine functionality to form an amide bond. It is to be recognized that a free amine may be introduced into a scaffold core in a number of ways. For example, the amine functionality may constitute the ε-NH₂-side chain of a lysine residue within a linear polypeptide scaffold. Alternatively, the free CO₂H-end of a peptide may be coupled to an amine functionality, for example, the free NH₂-end of a peptide scaffold. Alternative methods for forming amide bonds within the amino acid sequence of an N-peptide are available, which methods are known in the art.

In principle, the bond between N-peptide and scaffold core can be formed anywhere within an immunogenic N-peptide amino acid sequence, as long as the primary, secondary and tertiary sequence of the epitope of interest is essentially maintained. In one preferred embodiment a linkage is formed between any one of the ten N-terminal and ten C-terminal amino acid residues of the amino acid sequence. Preferably, a linkage is formed between any one of the six N-terminal and six C-terminal amino acid residues, preferably between any one of the four N-terminal and four C-terminal amino acid residues, of the amino acid sequence. Of course, the sites that are suitable for the formation of an internal bond are dependent on the location of the epitope(s) of interest. In one preferred embodiment a linkage is formed between the first and the last amino acid residue of an immunogenic amino acid sequence.

Given the aforementioned importance of maintaining optimal spacing of the N-peptide constituents to allow trimer formation, it will be recognized that the point of attachment to the scaffold may be direct or may be modified by the addition of linker molecules that increase the distance between the N-peptide and the core constituent of the scaffold. Linkers comprise any combination of atoms that may include but are not limited to carbon, nitrogen, oxygen, phosphorous and sulfur with lengths up to 50 atoms. Generally, the spacer/linker of the present invention may include any molecule that can bind and position three N-peptides at a sufficient distance to allow trimerization of the N-peptides to present a conformationally correct mimetic of the gp41 prehirpin intermediate and which presents the D5 neutralizing epitope in its native conformation.

A linker may be homobifunctional wherein the same reactive functional group is present at both ends of the molecule. Examples include but are not limited to 1,2 diaminoethane; 1,3 diaminopropane; putrescine; cadaverine; oxalic acid, malonic acid, succinic acid, adipic acid, 3,3′-dithiobis(sulfosuccinimidylpropionate); disuccinimidyl suberate; ethylene glycolbis(succinimidylsuccinate); dimethyl adipimidate; bismaleimidohexane; 1,5-difluoro-2,4-dinitrobenzene; adipic acid dihydrazide; carbohydrazide; and N,N′-ethylene-bis(iodoacetamide).

In a preferred embodiment of the instant invention, the carboxylic acid functionality of the Kemp's triacid scaffold is modified by reaction with the homobifunctional molecule diaminoethane to increase the distance from the cyclohexane ring and the N-peptide.

Alternatively, a linker may be heterobifunctional wherein a different reactive functional group is present at either end of the molecule. A wide variety of such molecules are readily available, classes of which include amine/sulfhydryl-reactive, carbonyl/sulfhydryl-reactive, amine/photo-reactive, sulfhydryl/photo-reactive, carbonyl/photo-reactive, and others. Specific examples include but are not limited to sulfosuccinimidyl 4-(N-maleidomethyl)-cyclohexane-1-carboxylate; 4-succinimidyloxycarbonyl-α-methyl-α-(2-pyridylditio)toluene; m-maleimidobenzoyl-N-hydroxysuccinide ester; sulfosuccinimidyl(4-iodoacetyl)-aminobenzoate; succinimidyl 4-(p-maleimidophenyl)butyrate; 3-(2-pyridyldithio)propionyl hydrazide; N-hydroxysuccinimidyl-4-azidosalicylic acid; benzophenone-4-iodoacetamide; p-azidobenzoyl hydrazide; and 5-aminopentylmaleimide. Heterobifunctional polyethylene glycol (PEG) linkers of varying length may also be employed and examples of specific classes include but are not limited to N-hydroxysuccinimidyl-PEG₍ₙ₎-maleimide; N-hydroxysuccinimidyl-PEG₍ₙ₎azide; and N-hydroxysuccinimidyl-PEG₍ₙ₎-propargyl.

In a preferred embodiment of the instant invention, an allylic-derivative of cholic acid is reacted with 2-aminoethanethiol and subsequently with γ-maleimidobutyric acid to increase the distance from the cholesterol ring and the N-peptide.

The N-peptide portion of the peptide mimetics described herein can be produced by a variety of methods. For example, they can be chemically synthesized. Long peptides may be synthesized on solid-phase supports using an automated peptide synthesizer as described by Kent et al., 1985, “Modern Methods for the Chemical Synthesis of Biologically Active Peptides,” Alitalo et al. (Eds.), Synthetic Peptides in Biology and Medicine, Elsevier pp. 29-57. Manual solid-phase synthesis may be performed as described, for example, in Merrifield, 1963, Am. Chem. Soc. 85:2149, or known improvements thereof. Solid-phase peptide synthesis may also be performed by the Fmoc method, which employs very dilute base to remove the Fmoc protecting group. Solution-phase synthesis is usually feasible only for selected smaller peptides. For preparing cocktails of closely related peptides, see, e.g., Houghton, 1985, Proc. Natl. Acad. Sci. USA 82:1242-1246. The peptide mimetics can be produced as a continuous peptide or as components that are joined or linked after they are formed.

Alternatively, the peptides described herein can be produced, using known methods and expression systems, by expressing chimeric peptide-encoding DNA, which can be a single DNA that encodes the entire chimeric peptide. The chimeric peptide gene may be recombinantly expressed by molecular cloning into an expression vector (e.g., pcDNA3.neo, pcDNA3.1, pCR2.1, pBlueBacHis2 or pLITMUS28) containing a suitable promoter and other appropriate transcription regulatory elements, and transferred into prokaryotic or eukaryotic host cells to produce the chimeric peptide. Expression vectors are defined herein as DNA sequences that are required for the transcription of cloned DNA and the translation of their mRNAs in an appropriate host. Such vectors can be used to express recombinant DNA in a variety of recombinant host cells such as bacteria, yeasts, blue green algae, plant cells, insect cells and mammalian cells. An appropriately constructed expression vector should contain the following components: an origin of replication for autonomous replication in host cells; selectable markers; a limited number of useful restriction enzyme sites; and active promoters. Expression vectors may include, but are not limited to, cloning vectors, modified cloning vectors, specifically designed plasmids or viruses. Commercially available mammalian expression vectors may be suitable for recombinant peptide expression. Also, a variety of commercially available bacterial, fungal cell, and insect cell expression vectors may be used to express recombinant mimotopes in the respective cell types. A promoter is defined as a DNA sequence that directs RNA polymerase to bind to DNA and initiate RNA synthesis. A strong promoter is one which causes mRNAs to be initiated at high frequency. Techniques for such manipulations can be found in Sambrook et al., 1989, Molecular Cloning: A Laboratory Manual, Cold Spring Harbor Laboratory, Cold Spring Harbor, N.Y., and are well known and available to an artisan of ordinary skill in the art. The expression vector containing the appropriate gene coding for a N-peptide may be introduced into host cells via any one of a number of techniques, including but not limited to transformation, transfection, protoplast fusion, and electroporation. The expression vector-containing cells are individually analyzed to determine whether they produce the peptide of interest. Identification of peptide-expressing cells may be done by several means, including but not limited to immunological reactivity with anti-HIV peptide antibodies. Recombinant peptides may possess additional and desirable structural modifications not shared with the same organically synthesized-peptide, such as adenylation, carboxylation, glycosylation, hydroxylation, methylation, phosphorylation or myristoylation. These added features may be chosen or preferred as the case may be, by the appropriate choice of recombinant expression system.

Following expression of a N-peptide gene in a host cell, N-peptide may be recovered. Several protein purification procedures are available and suitable for use, including purification from cell lysates and extracts, or from conditioned culture medium, by various combinations of, or individual application of, salt fractionation, ion exchange chromatography, reversed-phase chromatography, size exclusion chromatography, hydroxylapatite adsorption chromatography and hydrophobic interaction chromatography. In addition, peptides can be separated from other cellular proteins by use of an immunoaffinity column made with monoclonal or polyclonal antibodies specific for the peptide.

Certain peptides described herein, including (CCIZN36)₃ and 5-Helix, have been published. See, for example, Root et al., 2003, Proc Natl Acad Sci USA 100:5016-5021; Root et al., 2001, Science 291:884-888; Steger et al., 2006, Journal Biol Chem 281:25813-25821; Wang et al., 2009, Sheng wu gong cheng xue bao=Chinese journal of biotechnology 25:435-440; Bianchi et al., 2005, Proc Natl Acad Sci USA 102:12903-12908; Bianchi et al., 2009, Advances in experimental medicine and biology 611:121-123; Bianchi et al., Proc Natl Acad Sci USA 107:10655-10660; Eckert et al., 2001, Proc Natl Acad Sci USA 98:11187-11192; Eckert et al., 2001, Annual review of biochemistry 70:777-810; Eckert et al., 1999, Cell 99:103-115; Hrin et al., 2008, AIDS research and human retroviruses 24:1537-1544; Luftig et al., 2006, Nature structural & molecular biology 13:740-747; and Montgomery et al., 2009, mAbs 1:462-474. A number of publications have been identified which describe alternative ways for constraining NHR-based peptides as the basis for reverse-engineered vaccines. See, for example, Bomsel et al., Immunity 34:269-280; Chen et al., JBiol Chem 285:25506-25515; Corti et al., PloS one 5:e8805; de Rosny et al., J Virol 75:8859-8863; Dwyer et al., 2008, Protein Sci 17:633-643; Gokulan et al., 1999, DNA and cell biology 18:623-630; Korazim et al., 2006, J Molec Biol 364:1103-1117; Li et al., 2009, Immunobiology 214:51-60; Louis et al., 2001; JBiol Chem 276:29485-29489; Lu et al., J Pept Sci 16:465-472; Nelson et al., 2008, Virology 377:170-183; Pan et al., Journal of the Formosan Medical Association=Taiwan yi zhi 109:94-105; Qi et al., Biochem Biophys Res Comm 398:506-512; Qiao et al., 2005, JBiol Chem 280:23138-23146; Sabin et al., PLoS pathogens 6:e1001195; Sadler et al., 2008, Biopolymers 90:320-329; Schuy et al., 2009, J Structural Biol 168:125-136; Wexler-Cohen et al., 2009, PLoS pathogens 5:e1000509; Zhang et al., 2009, Vaccine 27:857-863.

It is contemplated that the conformationally constrained coiled-coil structures generated by the methods described herein encompass both homotrimeric coiled-coil structures (i.e., comprised of three identical N-peptides) or heterotrimeric coiled-coil structures (i.e., comprised of three N-peptides which are not identical, although substantially similar). In one embodiment, the heterogeneity of the heterotrimeric coiled-coil structures of the pepetide mimetics described herein may result from amino acid differences residing in the stabilizing region of the individual N-peptides comprising the coiled-coil structure. The heterogeneity of the heterotrimeric coiled-coil structures of the peptide mimetics described herein may result from amino acid differences residing within the individual N-peptides comprised within the coiled-coil. For example, a heterotrimeric coiled-coil structure may be comprised of three N-peptides wherein the “a” and “d” amino acid positions of the heptad repeat of each individual peptide, important for the trimerization ability of the peptides, are identical while the amino acid positions external to the hydrophobic region (e.g., position “f”) are different among the individual peptides of the trimeric coiled-coil. Importantly, such heterotrimeric structures could still be identified as faithful mimetics of a HIV gp41 fusion intermediate because the function of the coiled-coil is similar to that of the wildtype structure (e.g., antiviral activity and/or generation of a faithful conformation epitope).

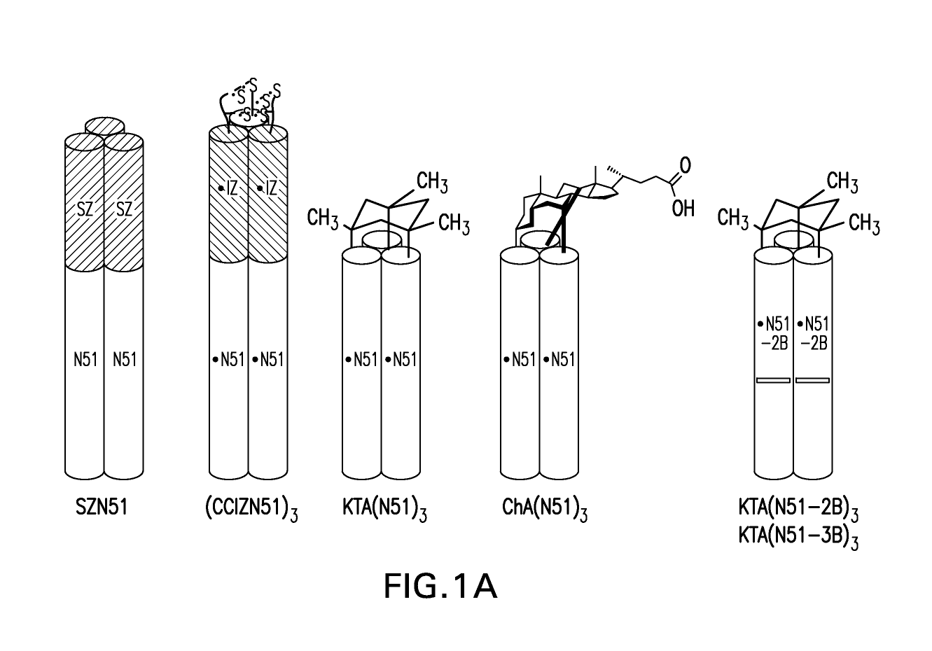

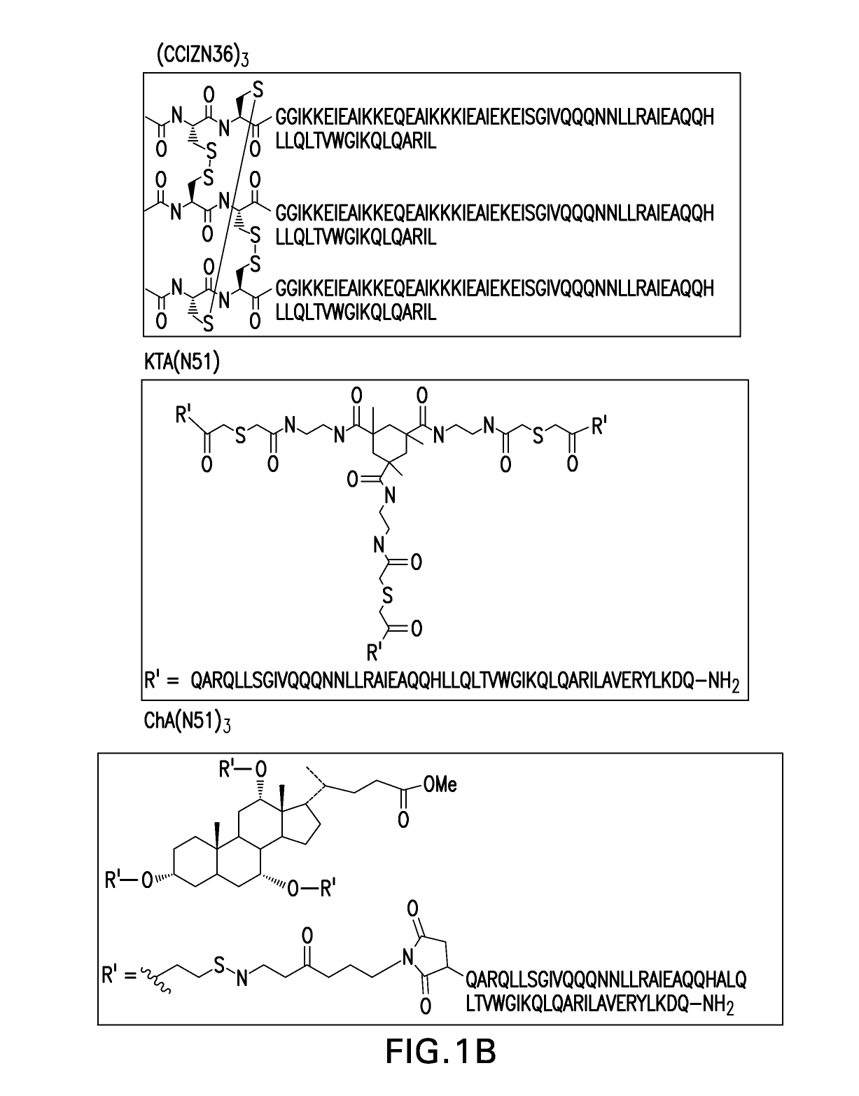

Trimeric structures of representative gp41 peptide mimetics are shown schematically in FIGS. 1A-B.

One of skill in the art can easily determine whether a resulting CC-chimeric N-peptide faithfully displays the N-peptide domain when in its trimeric, covalently-stabilized conformation, e.g., by testing either its ability to inhibit HIV infectivity with high potency or its capacity to bind an antibody that recognizes a conformational epitope located in the N-helix region of gp41. A number of different experimental methods can be used to determine whether or not a gp41 peptide mimetic can form a stable, faithful mimetic of said internal coiled-coil. For example, an assay designed to measure the ability of the conformationally constrained gp41 peptide mimetics to inhibit infectivity of HIV particles can be performed. In one such assay, HeLa cells stably expressing human CD4 and CCR5 receptors and harboring a β-galactosidase reporter gene driven by a tat-responsive fragment of HIV-2 LTR are infected with HIV-1 of various strains in the presence of gp41 peptide mimetics at varying concentrations. After incubating said cells for a specific period of time, the cells are lysed and β-galactosidase activity is quantified. If a gp41 peptide mimetic retains the ability to inhibit HIV infectivity by interfering with the gp41 fusion intermediate, a low β-galactosidase activity is recorded.

Since the gp41 peptide mimetics described herein represent stable, faithful mimetics of the internal, N-helix coiled-coil of gp41, they are believed to be useful as immunogens to raise a neutralizing antibody response targeting HIV fusion intermediates. The gp41 peptide mimetics, when administered, will likely offer a prophylactic advantage to previously uninfected individuals and/or provide a therapeutic effect by reducing viral load levels within an infected individual, thus prolonging the asymptomatic phase of HIV infection.

The peptide mimetics described herein can be administered by one or more of a variety of route(s), such as, nasally, intraperitoneally, intramuscularly, intravenously, vaginally or rectally. In each embodiment, the peptide mimetic is provided in an appropriate carrier or as an immunogenic composition. For example, a peptide mimetic can be administered in an appropriate buffer, saline, water, gel, foam, cream or other appropriate carrier. An immunogenic composition comprising the peptide mimetic and, generally, an appropriate carrier and optional components, such as stabilizers, absorption or uptake enhancers, and/or emulsifying agents, can be formulated and administered in prophylactically effective dose(s) to an individual (uninfected or infected with HIV). In one embodiment, peptide mimetics can be administered (or applied) as microbicidal agents and interfere with viral entry into cells. For example, a peptide mimetic can be included in a composition which is applied to or contacted with a mucosal surface, such as the vaginal, rectal or oral mucosa. The composition comprises, in addition to the peptide mimetic, a carrier or base (e.g., a cream, foam, gel, other substance sufficiently viscous to retain the peptide mimetic, water, buffer) appropriate for application to a mucosal surface or to the surface of a contraceptive device (e.g., condom, cervical cap, diaphragm). The peptide mimetic can be applied to a mucosal surface, such as by application of a foam, gel, cream, water or other carrier containing the peptide mimetic. Alternatively, it can be applied by means of a vaginal or rectal suppository which is a carrier or base which contains the peptide mimetic and is made of a material which releases or delivers the peptide mimetic (e.g., by degradation, dissolution, other means of release) under the conditions of use (e.g., vaginal or rectal temperature, pH, moisture conditions). In all embodiments, controlled or time release (gradual release, release at a particular time after administration or insertion) of the peptide mimetic can be effected by, for example, incorporating the peptide mimetic into a composition which releases the drug gradually or after a defined period of time. Alternatively, the peptide mimetic can be incorporated into a composition which releases the peptide mimetic immediately or soon after its administration or application (e.g., into the vagina or rectum). Combined release (e.g., release of some of the drug immediately or soon after insertion, and over time or at a particular time after insertion) can also be effective (e.g., by producing a composition which is comprised of two or more materials: one from which release or delivery occurs immediately or soon after insertion and/or one from which release or delivery is gradual and/or one from which release occurs after a specified period). For example, a peptide mimetic can be incorporated into a sustained release composition such as that taught in U.S. Pat. No. 4,707,362. The cream, foam, gel or suppository can be one also used for birth control purposes (e.g., containing a spermicide or other contraceptive agent), although that is not necessary (e.g., it can be used solely to deliver the peptide mimetic, alone or in combination with another non-contraceptive agent, such as an antibacterial or antifungal drug or a lubricating agent). A peptide mimetic of the present invention can also be administered to an individual through the use of a contraceptive device (e.g., condom, cervical cap, diaphragm) which is coated with or has incorporated therein in a manner which permits release under conditions of use a peptide mimetic. Release of the peptide mimetic can occur immediately, gradually or at a specified time, as described above. As a result, they make contact with and bind HIV and reduce or prevent viral entry into cells.

In general, selection of the appropriate “effective amount” or dosage for the components of the immunogenic compositions of the present invention will also be based upon the identity of the peptide mimetic in the immunogenic composition(s) employed, as well as the physical condition of the subject, most especially including the general health, age and weight of the immunized subject. The method and routes of administration and the presence of additional components in the immunogenic compositions may also affect the dosages and amounts of the compositions. Such selection and upward or downward adjustment of the effective dose is within the skill of the art. The amount of composition required to induce an immune response, preferably a protective response, or produce an exogenous effect in the subject without significant adverse side effects varies depending upon these factors. Suitable doses of the immunogenic compositions described herein are readily determined by persons skilled in the art. A dose of a gp41 peptide mimetic sufficient to reduce HIV infection (an “effective dose”) is administered in such a manner (e.g., by injection, topical administration, intravenously) that it inhibits, totally or partially, HIV entry into cells. Dosages of between 10 mg and 1000 mg of gp41 peptide mimetic, and preferably between 50 mg and 300 mg of peptide mimetic, are administered to a mammal to induce anti-HIV or HIV-neutralizing immune responses. In one embodiment, the peptide mimetic should be given intramuscularly at a concentration of between 10 μg/ml and 1 mg/ml, and preferably between 50 and 500 μg/ml, in a volume sufficient to make up the total required for immunological efficacy.

In some embodiments of the invention, the peptide mimetics of the invention can be used in a prime boost regimen. Priming components for such an approach may include but need not be restricted to DNA, genetic vectors, peptides, or proteins. Such regimen can be homologous or heterologous. For example, about two to four weeks after the initial administration, a booster dose (whether homologous or heterologous) may be administered, and then again whenever serum antibody titers diminish. Multiple prime administrations may also be used, followed by two to four weeks after the last prime administration. A heterologous boost can involve peptide mimetics which differ from the peptide mimetic used for the prime. A heterologous boost can also involve other HIV prophylactics known in the art such as recombinant gp120, gp 140, and gp160 molecules administered as either DNA or protein components.

In some embodiments of the invention, the peptide mimetics described herein can be covalently conjugated to an immunogenic carrier protein, for example, to enhance the immune response to the peptide mimetic. Such bioconjugation approaches are well known to those skilled in the art and it will be recognized that a variety of carrier proteins and conjugation chemistries may be employed.

An immunogenic composition suitable for patient administration will contain an effective amount of the peptide mimetic in a formulation which both retains biological activity while also promoting maximal stability during storage within an acceptable temperature range. An immunogenic composition comprising the peptide mimetics either in the priming or boosting dose in accordance with the instant invention may contain physiologically acceptable components, such as buffer, normal saline or phosphate buffered saline, sucrose, other salts and polysorbate. One skilled in the art will appreciate that other conventional vaccine excipients may also be used it make the formulation. Adjuvants may or may not be added during the preparation of immunogenic compositions containing the peptide mimetics described herein. For example, alum is the typical and preferred adjuvant in human vaccines, especially in the form of a thixotropic, viscous, and homogeneous aluminum hydroxide gel.

These peptide mimetics could be used in combination with a variety of anti-retrovirals to inhibit HIV replication and/or other HIV proteins. Classes of anti-retrovirals that could be used with peptide mimetics-based compositions include, but are not limited to, nucleoside reverse transcriptase inhibitors (NRTIs), non-nucleoside reverse transcriptase inhibitors (NNRTIs) and protease inhibitors (PIs). Other HIV proteins include gp120, gp140, and gp160. DNA vectors encoding HIV proteins are also suitable and may encode HIV proteins including, but not be limited to, gp120, gp140, and gp160 molecules.

In certain embodiments, the present invention provides a kit for administration of the regimens described herein. This kit is designed for use in a method of inducing a immunogenic response in a mammalian or vertebrate subject. The kit contains an immunogenic composition comprising a gp41 peptide mimetics of the invention. Preferably multiple prepackaged dosages of the immunogenic composition are provided in the kit for multiple administrations.

The kit also contains instructions for using the immunogenic compositions as described herein. The kits may also include instructions for performing certain assays, various carriers, excipients, diluents, adjuvants and the like above-described, as well as apparatus for administration of the compositions, such as syringes, spray devices, etc. Other components may include disposable gloves, decontamination instructions, applicator sticks or containers, among other compositions.

Having described preferred embodiments of the invention with reference to the accompanying figures, it is to be understood that the invention is not limited to those precise embodiments, and that various changes and modifications may be effected therein by one skilled in the art without departing from the scope or spirit of the invention as defined in the appended claims.

The following non-limiting Examples are presented to better illustrate the invention.

### Example 1

### Immunogen Production and Characterization

### Immunogen Production: Synthetic Peptides

### 1. (CCIZN36)₃

The peptide monomer CCIZN36 (CCGGIKKEIEAIKKEQEAIKKKIEAIEKEISGIVQQQNNLLRAIEAQQHLLQLTVWGIK QLQARIL (SEQ ID NO:46) was synthesized using solid phase Fmoc/t-Bu chemistry on an automated peptide synthesizer. The resin used was H-Rink Amide ChemMatrix (Matrix-Innovation Inc., St. Hubert, Quebec, Canada). Acylations were performed with double couplings of 30 minutes each cycle using a 5-10 fold excess of amino acids over resin free amino groups Amino acids were activated with an equimolar amount of HATU [2-(1H-9-Azabenzotriazole-1-yl)-1,1,3,3-tetramethyl-aminum hexafluorophosphate] and a 2-fold molar excess of DIEA (N,N-diisopropylethylamine). The side chain protecting groups used were as follows: trityl for Cysteine, Glutamine, Asparagine, and Histidine; tert-butoxy-carbonyl for Lysine and Tryptophan; tert-butyl for Glutamic acid, Threonine, and Serine; and 2,2,4,6,7-pentametyldihydrobenzofuran-5-sulfonyl for Arginine. At the end of the synthesis, the peptide was cleaved from the resin by treatment with 90% trifluoroacetic acid, 5% triisopropylsilane, 2.5% water, and 2.5% 3,6-dioxa-1,8-octane-dithiol for 3 hours at room temperature. The peptide solution was filtered and added to cold diethyl ether to precipitate the peptide. The precipitated peptide was pelleted by centrifugation, and the pellets were then washed twice with cold diethyl ether to remove organic scavengers. The final pellets were dried, re-suspended in 25% acetic acid in water, and lyophilized.

The crude peptide was purified by reverse phase HPLC using a Jupiter C18 column (250×30 mm, 10μ, 300 A, Phenomenex, Inc., Torrance, Calif.) with a water/acetonitrile gradient in the presence of 0.1% trifluoroacetic acid. The purified peptide was characterized by electrospray mass spectrometry. The monoisotopic mass determined for the purified peptide was 7541.22 Da (the sequence-predicted mass is 7542.24 Da).

Purified CCIZN36 (35 mg) was dissolved in 30 ml of buffer (pH 7.5) containing 1 N guanidine, 0.2M HEPES, 1 mM EDTA, 1.5 mM reduced glutathione, and 0.75 mM oxidized glutathione. Under these conditions, CCIZN36 is slowly oxidized to the covalent trimer form of the molecule (CCIZN36)₃. The progress of the oxidation reaction was monitored by HPLC, and after 24 hours, the reaction was terminated by the addition of 500 μl of trifluoroacetic acid to the reaction mixture which was directly loaded on a Vydac® diphenyl column (22×250 mm, 10-15μ, Grace, Deerfield, Ill.) and purified by reverse phase HPLC using a water/acetonitrile gradient in the presence of 0.1% trifluoroacetic acid at a flow rate of 20 ml/min. Fractions were analyzed by RP HPLC/mass spectrometry. The fractions corresponding to the covalent trimer were pooled for further use. The monoisotopic mass determined for the purified trimer was 22620.58 Da (the sequence-predicted mass is 22620.681 Da).

### 2. KTA(N51)₃

The trimeric peptide complex was synthesized through ligation of three thiol tagged monomer N51 peptides, S-acetylglycolic-N51, on a trivalent bromide scaffold, KTA-Br.

The KTA-Br is a Kemp's triacid-centered symmetric trivalent scaffold. It was synthesized according to the protocol described in Xu et al., 2007, Chem Bio Drug Des 70:319-328, with the modification that bromoacetic anhydride was used during the final acylation step.

The peptide N51 was synthesized by solid phase using Fmoc/t-Bu chemistry on an automated peptide synthesizer. The resin used was H-Rink Amide ChemMatrix (Matrix-Innovation Inc.), a 100% PEG resin. Acylations were performed with double couplings for 30 minutes with 5-10 fold excess of amino acids over the resin free amino groups. Amino acids were activated with equimolar amounts of HATU [2-(1H-9-Azabenzotriazole-1-yl)-1,1,3,3-tetramethyl-aminum hexafluorophosphate] and a 2-fold molar excess of DIEA (N, N-diisopropylethylamine). The side chain protecting groups were as follows: trityl for Glutamine, Asparagine, and Histidine; tert-butoxy-carbonyl for Lysine and Tryptophan; tert-butyl for Glutamic acid, Threonine, and Serine; and 2, 2,4,6,7-pentametyldihydrobenzofuran-5-sulfonyl for arginine. A protected thiol group was introduced to the peptide N terminus by manually coupling of S-acetylthioglycolic acid pentafluorophenyl ester (SAMA-OPfp) in the presence of equal amount of N-hydroxybenzotriazole, at the end of sequence assembly. The acetyl group that protects the thiol can be easily removed by hydroxylamine during the next ligation step. At the end of the synthesis, the dry peptide resin was treated with cleavage mixture (95% trifluoroacetic acid, 2.5% triisopropylsilane, 2.5% water) for 3 hours at room temperature. The resin was filtered and the solution was added to cold diethyl ether in order to precipitate the peptide. After centrifugation, the peptide pellets were washed twice with cold diethyl ether to remove organic scavengers. The final pellets were dried, re-suspended in 25% acetic acid in water, and lyophilized.

The crude peptide was purified by reverse phase HPLC using a Jupiter C18 column (250×30 mm, 10μ, 300 A), and a water/acetonitrile gradient in the presence of 0.1% trifluoroacetic acid at a flow rate is 40 ml/min. Analytical HPLC was performed on a Jupiter C18 column (150×4.6 mm, 5μ, 300 A). The purified peptide was characterized by electrospray mass spectrometry. The ESI spectrum shows charge status +4 to +7. The deconvoluted mass is 6051.6 Da (the sequence-predicted mass is 6051 Da).

The purified peptide precursor S-acetylglycolic-N51 (60 mg) was dissolved in 8 ml of pH 7-7.5 buffer that contains 6 N guanidine, 0.1 N ammonium acetate, and 0.5 N hydroxylamine 1.7 mg of KTA-Br₃ was dissolved in 1 ml of trifluoroethanol, and was then added drop wise to the peptide solution. The reaction was monitored by LC-MS. After 5 hours, the reaction was terminated by adding 500 μl of TFA to the solution, and the solution was directly loaded on a Vydac® diphenyl column (22×250 mm, 10-15μ) and purified by reverse phase HPLC using a water/acetonitrile gradient in the presence of 0.1% trifluoroacetic acid at flow rate of 20 ml/min. The pooled fractions corresponding to the covalent trimer were further analyzed by mass spectrometry. The ESI spectrum shows charge status +14 to +18. The deconvoluted mass is 18532.8 Da (the sequence-predicted mass is 18532 Da).

### 3. KTA(N51-2B)₃

The trimeric peptide complex was synthesized via ligation of three thiol tagged monomeric N51-2B peptides, S-acetylglycolic-(N51-2B), on a trivalent bromide scaffold, KTA-Br by following the same protocol as described for synthesis of KTA(N51)₃.

The N51-2B sequence was designed to attempt to produce a more soluble and stabilized N51 trimer. Ile residues were added at the “a” and “d” positions to optimize trimer formation and other substitutions were made at “f” and “c” positions in the N terminal part of the peptide to aid in solubility. One Leu residue was changed to an Ala residue near the hydrophobic pocket, but the Leu that was part of hydrophobic pocket was maintained.

The peptide N51-2B was synthesized by solid phase using Fmoc/t-Bu chemistry on an automated peptide synthesizer. The resin used was H-Rink Amide MBHA resin LL (100-200 mesh, 0.36 mmol/g) (EMD Biosciences, San Diego, Calif.). The synthesis, cleavage, and purification of S-acetylglycolic-N51-2B followed the same protocol as described for S-acetylglycolic-N51. The purified peptide was characterized by electrospray mass spectrometry. The ESI spectrum shows charge status +4 to +7. The deconvoluted mass is 6109 Da (the sequence-predicted mass is 6109 Da).

The purified peptide precursor S-acetylglycolic-N51 (10 mg) was dissolved in lml of pH 7.3 buffer that contains 6N guanidine, 0.1 N ammonium acetate, and 0.5 N hydroxylamine About 0.25 eq. of KTA-Br₃ was dissolved in trifluoroethanol, and was added drop wise to the peptide solution. The reaction was monitored by LC-MS. After 4 hours, the reaction was terminated, and the solution was directly loaded on a Vydac® C.18 column (10×250 mm, 5μ) and purified by reverse phase HPLC using a water/acetonitrile gradient in the presence of 0.1% trifluoroacetic acid at a flow rate of 5 ml/min. The pooled fractions corresponding to the covalent trimer were further analyzed by mass spectrometry. The ESI spectrum shows charge status +14 to +18. The deconvoluted mass is 18708.8 Da (the sequence-predicted mass is 18709 Da).

### 4. KTA (N51-3B)₃

The trimeric peptide complex was synthesized via ligation of three thiol tagged monomeric N51-3B peptides, S-acetylglycolic-(N51-3B), on a trivalent bromide scaffold, KTA-Br by following the same protocol as described for synthesis of KTA(N51)₃.

The N51-3B sequence was designed to attempt to produce a more soluble and stabilized N51 trimer. The sequence is a single change to Ala in the original N51.

The peptide N51-3B was synthesized by solid phase using Fmoc/t-Bu chemistry on an automated peptide synthesizer. The resin used was H-Rink Amide MBHA resin LL (100-200 mesh, 0.36 mmol/g) (EMD Biosciences). The synthesis, cleavage, and purification of S-acetylglycolic-N51-3B followed the same protocol as described for S-acetylglycolic-N51. The purified peptide was characterized by electrospray mass spectrometry. The ESI spectrum shows charge status +4 to +7. The deconvoluted mass is 6007.8 Da (the sequence-predicted mass is 6010 Da).

The purified peptide precursor S-acetylglycolic-N51 (5 mg) was dissolved in 1 ml of pH 7.3 buffer that contains 6 N guanidine, 0.1 N ammonium acetate, and 0.5 N hydroxylamine About 0.25 eq. of KTA-Br₃ was dissolved in trifluoroethanol, and was added dropwise to the peptide solution. The reaction was monitored by LC-MS. After 4 hours, the reaction was terminated, and the solution was directly loaded on a Vydac® C4 column (10×250 mm, 5μ) and purified by reverse phase HPLC using a water/acetonitrile gradient in the presence of 0.1% trifluoroacetic acid at a flow rate of 5 ml/min. The pooled fractions corresponding to the covalent trimer were further analyzed by mass spectrometry. The ESI spectrum shows charge status +14 to +18. The deconvoluted mass is 18405.4 Da (the sequence-predicted mass is 18406 Da).

### 5. Cholic Acid (N51)₃

The trimeric peptide complex was synthesized via ligation of three thiol tagged monomeric N51 peptides, S-acetylglycolic-N51, on a trivalent trimaleimido cholic acid template.

Cholic acid sodium 1 (2 g, 4.65 mmole) was suspended in 25 ml of THF (tetrahydrofuran). Allyl iodide (3.8 ml, 1 eq) was added in the mixture, followed by adding 2.2 g of sodium hydride (60%). The resulting mixture was stirred at 70° C. for overnight and was monitored by TLC and LC-MS. Then water was added to the reaction mixture, and ethylacetate/1 N HCl. Product was extracted into the organic layer, was washed with brine, and dried over anhydrous Na₂SO₄. The resultant crude product was characterized by LC-MS and was confirmed that the major component is the trivalent allyl cholic acid 2 with molecular weight of 528.7 Da.

Allyl cholic acid 2 (300 mg, 0.8 mmole), cysteamine hydrochloride, and azobisisobutyronitrile (AIBN) (as radical initiator) were mixed with methanol (5 ml, degassed with nitrogen) in a photo reactor. The mixture was irradiated with a UV lamp at 254 nm wavelength, and was stirred at room temperature over three days. Reaction was monitored by LC-MS and TLC. Products of compound 3 (C₃₉H₇₃N₃O₅S₃, MW=760.29), and its methyl ester 4 (C₄₀H₇₅N₃O₅S₃ MW=774.25) were yielded in 1:1 ratio.

Compound 4 (10 mg, 1 eq) was dissolved in 1 ml of DMF, then γ-maleimidobutyric acid (3.6 eg), HATU (1 eq), and triethylamine (2 eq) were added. The reaction was stirred for 2 hours to completion. After evaporation, the residue was extracted by ethyl acetate/1 N HCl, NaHCO₃, and brine. The organic layer was dried over Na₂SO₄ and filtered. The filtrate was concentrated, then purified to yield compound 5 with molecular weight of 1269.6 Da; while the found (M⁺Na⁺) peak is 1292.26 Da.

The purified peptide precursor S-acetylglycolic-N51 (4.8 mg) was dissolved in 0.2 ml of pH 7 buffer that contains 20 mM Tris and 0.5 N hydroxylamine 50 μg of trimaleimido cholic acid was dissolved in 50 μl of DMF/TFE, and added dropwise to the peptide solution. The reaction was monitored by LC-MS. After 2 hours, the reaction was complete, and the mixture was directly loaded on a Jupiter C18 column (10×250 mm, 10μ) and purified by reverse phase HPLC using a water/acetonitrile gradient in the presence of 0.1% trifluoroacetic acid at a flow rate of 5 ml/min. The pooled fractions corresponding to the covalent trimer compound 6 cholic acid (N51)₃ (ChA-(N51)₃) were further analyzed by mass spectrometry. The theoretical average molecular weight is 19296 Da, while the found monoisotopic peak is 19289.6 Da.

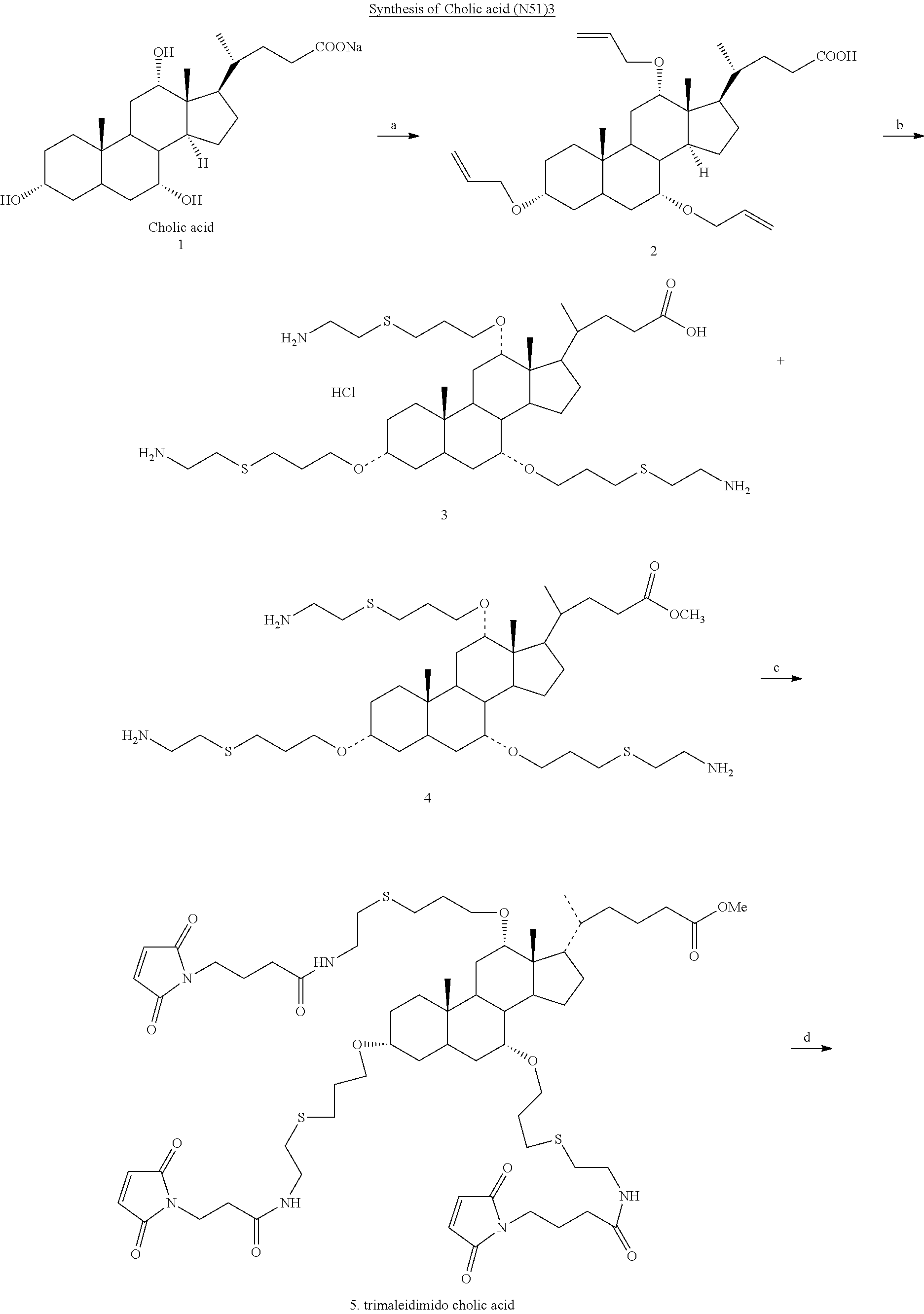

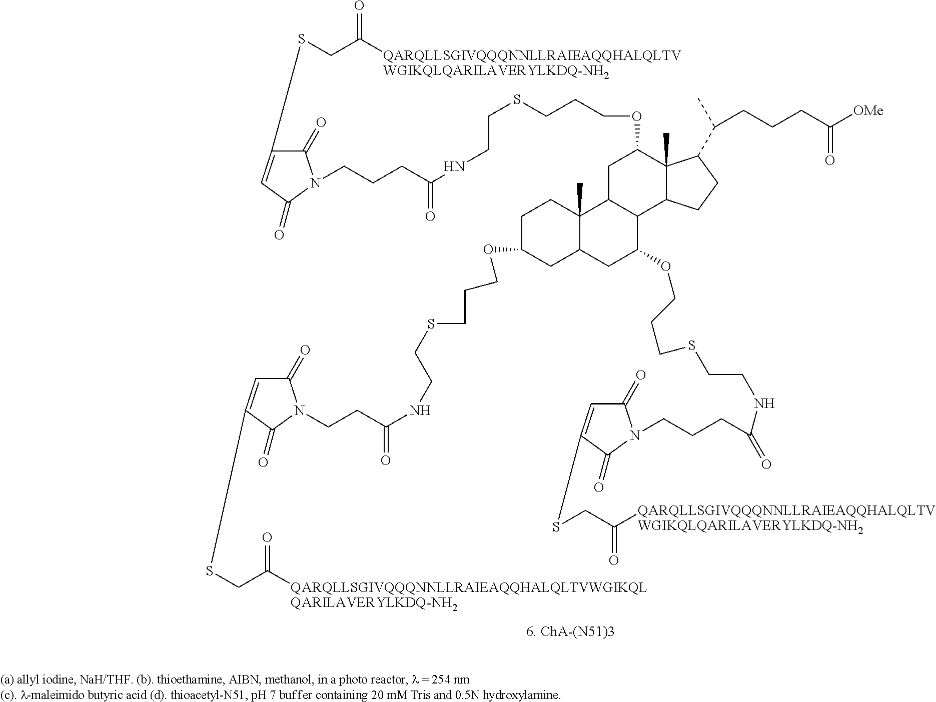

6. Cholic Acid (N51)₃ with a Thiol for Conjugation

The trimeric peptide complex was synthesized via ligation of three thiol tagged monomeric N51 peptides, thiopropanoic acyl-N51, on a trimaleimido cholic acid template with a masked thiol group. After ligation, the masked thiol is removed to form the conjugatable cholic acid-(N51)₃.

The mixture of cysteamine (1.15 g) and acetamidemethanol (1 g) were dissolved in TFA (triflouroacetic acid), and were stirred at room temperature for 3 hours to yield (S-acetamidomethyl) cysteamine 7 with molecular weight of 148 Da; while the found (M+H) ion peak is 149 Da.

Allyl cholic acid 2 (100 mg) was mixed with EDC (ethylene dichloride), DIEPA (N,N-Diisopropylethylamine) in DCM (dichloromethane), followed by addition of compound 7 (56 mg) dissolved in DCM. The reaction was monitored by TLC and found to be completed in 2 hours. After dilution of the reaction mixture with DCM, 1 N HCl was added, and the compound was extracted into the organic layer. The organic layer was dried over Na₂SO₄, and concentrated to yield compound 8 with molecular weight of 658.97 Da; while the found (M+H) ion peak is 659 Da.

Compound 8 (500 mg), cysteamine hydrochloride, and azobisisobutyronitrile (AIBN) were mixed with methanol (5 ml, degassed with nitrogen) in a photo reactor. The mixture was irradiated with a UV lamp at 254 nm, and was stirred at room temperature over three days. The reaction progress was monitored by LC-MS and TLC. Upon completion of reaction, water was added to the reaction mixture, followed by ethylacetate/1 N HCl (1:1) to obtain the product triaminocholic acid (compound 9), having a theoretical molecular weight of 889.5 Da; and an observed molecular weight of 890 Da ((M+H) ion peak).

Compound 9, (50 mg, 1 eq) was dissolved in 2 ml of DMF, then γ-maleimidobutyric acid (4.5 eq), HATU (4.5 eq), and triethylamine (9 eq) were added. The reaction was stirred for 2 hours to completion. After evaporation, the residue was extracted with ethyl acetate/1 N HCl, NaHCO₃, and brine. The organic layer was dried over Na₂SO₄, then filtered. The filtrate was concentrated and purified to yield compound 10, theoretical molecular weight 1384.66 Da. found (M+H) ion peak is 1385.6 Da.

The peptide N51 was synthesized by solid phase using Fmoc/t-Bu chemistry on an automated peptide synthesizer. The resin used was H-Rink Amide ChemMatrix (Matrix-Innovation Inc.). Acylations were performed with double couplings for 30 minutes with 5-10 fold excess of amino acids over the resin free amino groups Amino acids were activated with equimolar amounts of HATU [2-(1H-9-Azabenzotriazole-1-yl)-1,1,3,3-tetramethyl-aminum hexafluorophosphate] and a 2-fold molar excess of DIEA (N, N-diisopropylethylamine) The side chain protecting groups were as follows: trityl for Glutamine, Asparagine, and Histidine; tert-butoxy-carbonyl for Lysine and Tryptophan; tert-butyl for Glutamic acid, Threonine, and Serine; and 2,2,4,6,7-pentametyldihydrobenzofuran-5-sulfonyl for Arginine. After sequence assembly, the N-terminal amino group on peptide resin coupled to 3-(tritylthio) propionic acid in DMF in the presence of HATU and triethylamine At the end of the synthesis, the dry peptide resin was treated with cleavage mixture (92.5% trifluoroacetic acid, 2.5% triisopropylsilane, 2.5% water, and 2.5% 3,6-dioxa-1,8-octane-dithiol) for 2 hours at room temperature. The resin was filtered and the solution was added to cold diethyl ether in order to precipitate the peptide. After centrifugation, the peptide pellets were washed twice with cold diethyl ether to remove organic scavengers. The final pellets were dried, re-suspended in 25% acetic acid in water, and lyophilized.

The crude peptide was purified by reverse phase HPLC using a Jupiter C18 column (250×30 mm, 10μ, 300 A), a water/acetonitrile gradient in the presence of 0.1% trifluoroacetic acid at a flow rate of 40 ml/min. Analytical HPLC was performed on a Jupiter C18 column (150×4.6 mm, 5μ, 300 A). The purified peptide was characterized by electrospray mass spectrometry. The ESI spectrum shows charge status +4 to +7. The deconvoluted mass is 6022 Da (the sequence-predicted mass is 6023 Da).

Purified peptide thiopropionic N51 (15 mg) was dissolved in 5 ml of 20 mM Tris buffer (pH 7.0). The template compound 10 (1.05 mg) dissolved in 2 ml of acetonitrile solution was added to the peptide solution. The reaction was monitored by HPLC. After 1 hour, the resulting mixture was directly loaded on C18 column and purified to get product 11 with the theoretical average molecular weight of 19455 Da. The ESI spectrum shows charge status +13 to +18. The deconvoluted mass is 19451.5 Da.

Compound 11 (5 mg) was dissolved in 5 ml of pH 4 aqueous buffer containing acetic acid. Mercury acetate (2.1 mg) was dissolved in 2 ml of acetonitrile, and was added dropwise to the peptide solution. The reaction was monitored by HPLC. After 1 hour, 12 μl of 2-mecarptoethanol was added to the mixture. The resulting mixture was heated at 50° C. for 3 hours, followed by purification using a PD10 desalting column (GE Healthcare Lifesciences, Piscataway, N.J.) with 5% acetic acid as eluant. The ESI spectrum shows charge status +13 to +19. The deconvoluted mass is 19384 Da (the sequence-predicted mass is 19384 Da).

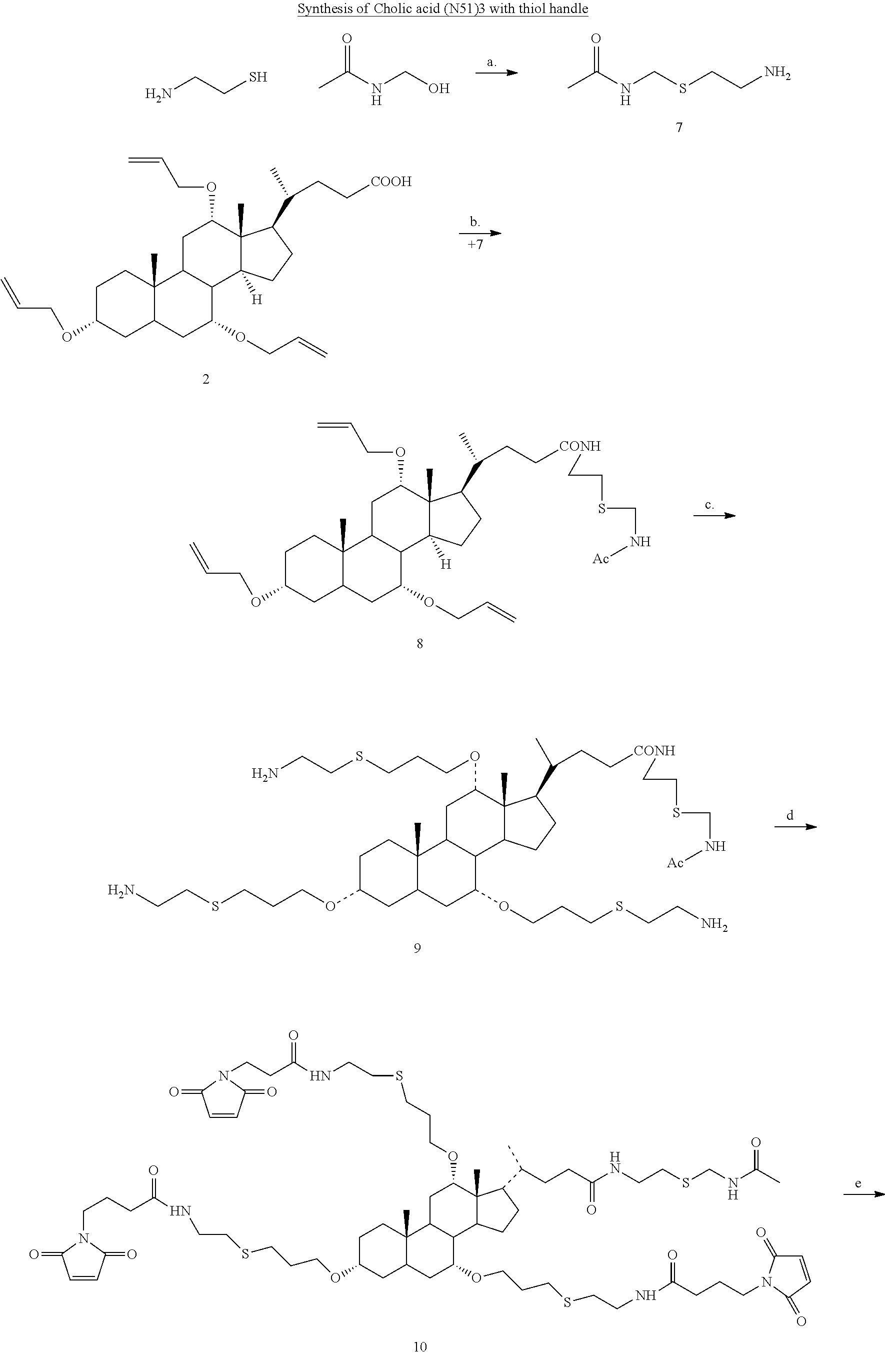

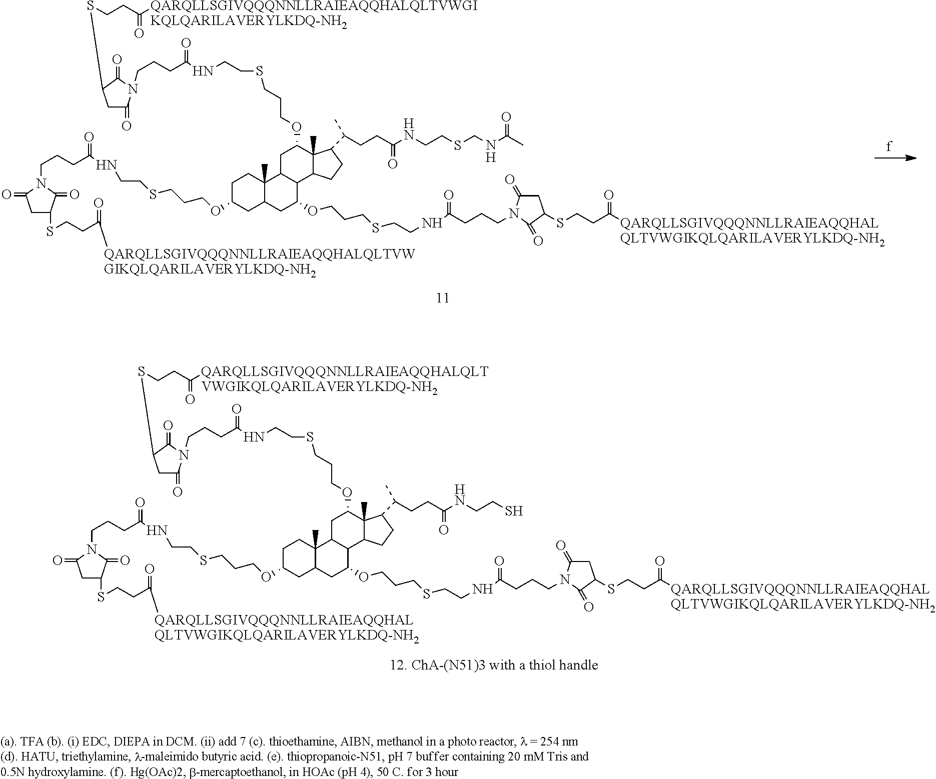

### Immunogen Production: Recombinant Peptides

### 1. Recombinant (CCIZN36)₃

A synthetic gene encoding the peptide sequence listed below was assembled from synthetic oligonucleotides and/or PCR products by GeneArt® (Life Technologies Corporation, Carlsbad, Calif.). The fragments were cloned into pET20b_A092 (EMD Biosciences, Gibbstown, N.J.) using NdeI and BamHI cloning sites. The plasmids were purified from transformed bacteria and concentration determined by UV spectroscopy. The final constructs were verified by sequencing. The sequence congruence within the used restriction sites was 100%. The plasmids were lyophilized prior to use.

CCIZN36:
(SEQ ID NO: 46)
CCGGIKKEIEAIKKEQEAIKKKIEAIEKEISGIVQQQNNLLRAIEAQQHL
LQLTVWGIKQLQARIL

BL21(DE3)pLysS competent cells (Invitrogen™, Life Technologies Corporation, Carlsbad, Calif.) were transformed with the plasmids encoding the gene for CCIZN36 according to the manufacturer's directions. The transformed cells were plated on Luria-Bertani (LB) agar with 50 μg/mL ampicillin and 34 μg/mL chloramphenicol and grown overnight at 37° C. Several colonies were picked from the plates, and LB media with antibiotics was inoculated with a single colony and grown overnight at 37° C. with shaking at 225 rpm. Glycerol stocks were also prepared for each colony from the overnight cultures, and the glycerol stocks were used as the starting material for future scale-up expression experiments. Fresh LB media with antibiotics was inoculated with a 1:40 dilution of the overnight preculture, and grown at 37° C. until the optical density at 600 nm reached between 0.6 and 0.8. Protein expression was then induced by addition of 0.5 mM isopropyl-b-D-thiogalactopyranoside (IPTG). The cultures were grown for an additional 3-4 hours at 37° C., and then the cells were harvested by centrifugation at 10,000×g for 15 minutes at 4° C. The cell pellets were stored at −20° C.

The cells were resuspended in lysis buffer (50 mM Tris, pH 8.0, 2 mM MgCl₂, 10 mM DTT, 70 U/mL Benzonase® (EMD Biosciences, Gibbstown, N.J.), 1× Roche Complete™ protease inhibitor cocktail (Roche Diagnostics Corp., Indianopolis, Ind.)) and lysed by 3 passes through the microfludizer. The lysate was then clarified by centrifugation. SDS-PAGE and western blot analysis confirmed that the majority of the CCIZN36 product was detected in the insoluble fraction. Accordingly, washed inclusion bodies were prepared from the insoluble fraction by repeated homogenization and centrifugation steps. The final washed inclusion bodies were pelleted by centrifugation and frozen at −70° C.

Purified inclusion bodies (2 g) were dissolved in 50% acetonitrile in water with 0.1% TFA, along with 200 mg of TCEP [(tris(2-carboxyethyl)phosphine]. The solution was maintained at room temperature overnight. The following morning the preparation was clarified by centrifugation. The supernatant containing the recombinant peptide was loaded onto a Jupiter C18 column (250×30 mm, 10μ, 300 A) and purified by reverse phase HPLC using a water/acetonitrile gradient in the presence of 0.1% trifluoroacetic acid at a flow rate of 40 ml/min. Analytical HPLC was performed on a Vydac® diphenyl column (150×4.6 mm), and the peptide was characterized by electrospray mass spectrometry. The ESI spectrum shows charge status +4 to +11. The deconvoluted mass is 7510.1 Da (the sequence-predicted mass is 7506 Da).

The purified peptide precursor (60 mg) was dissolved in 80 ml of buffer (pH 7.5) containing 1 N guanidine, 0.2 M HEPES, 1 mM EDTA, 1.5 mM of reduced form glutathione, and 0.75 mM of oxidized form glutathione. The oxidation reaction was monitored by HPLC. After overnight, the reaction was terminated by adding 500 μl of TFA to the solution, and the solution was directly loaded on a Vydac® diphenyl column (22×250 mm, 10-15μ) and purified by reverse phase HPLC using a water/acetonitrile gradient in the presence of 0.1% trifluoroacetic acid at a flow rate 20 ml/min. Analytical HPLC analysis was as the same as described above. The pooled fractions corresponding to the covalent trimer were further analyzed by high resolution mass spectrometry. The ESI spectrum shows charge status +12 to +27. The deconvoluted mass is 22524 Da (the sequence-predicted mass is 22511 Da).

### 2. Recombinant (CCIZN51)₃

A synthetic gene encoding the peptide sequence listed below was assembled from synthetic oligonucleotides and/or PCR products by GeneArt®. The fragments were cloned into pET20b_A092 using NdeI and BamHI cloning sites. The plasmids were purified from transformed bacteria and concentration determined by UV spectroscopy. The final constructs were verified by sequencing. The sequence congruence within the used restriction sites was 100%. The plasmids were lyophilized prior to use.

CCIZN51:
(SEQ ID NO: 40)
CCGGIKKEIEAIKKEQEAIKKKIEAIEKEIVQARQLLSGIVQQQNNLLRA
IEAQQHLLQLTVWGIKQLQARILAVERYLKDQ

BL21(DE3)pLysS competent cells (Invitrogen™) were transformed with the plasmids encoding the gene for CCIZN51 according to the manufacturer's directions. The transformed cells were plated on Luria-Bertani (LB) agar with 50 μg/mL ampicillin and 34 μg/mL chloramphenicol and grown overnight at 37° C. Several colonies were picked from the plates, and LB media with antibiotics was inoculated with a single colony and grown overnight at 37° C. with shaking at 225 rpm. Glycerol stocks were also prepared for each colony from the overnight cultures, and the glycerol stocks were used as the starting material for future scale-up expression experiments. Fresh LB media with antibiotics was inoculated with a 1:40 dilution of the overnight preculture, and grown at 37° C. until the optical density at 600 nm reached between 0.6 and 0.8. Protein expression was then induced by addition of 0.5 mM isopropyl-b-D-thiogalactopyranoside (IPTG). The cultures were grown for an additional 3-4 hours at 37° C., and then the cells were harvested by centrifugation at 10,000×g for 15 minutes at 4° C. The cell pellets were stored at −20° C.

The cells were resuspended in lysis buffer and lysed by 3 passes through the microfludizer. The lysate was then clarified by centrifugation. SDS-PAGE and western blot analysis confirmed that the majority of the CCIZN51 product was detected in the insoluble fraction. Accordingly, washed inclusion bodies were prepared from the insoluble fraction by repeated homogenization and centrifugation steps. The final washed inclusion bodies were pelleted by centrifugation and frozen at −70° C.

The inclusion bodies were dissolved in a buffer containing 4 M urea, 20 mM HEPES, and 20 mM of TCEP. The solution was kept at room temperature, overnight. After centrifugation, the supernatant was directly loaded on a Vydac® diphenyl column (22×250 mm, 10-15μ) and purified by reverse phase HPLC using a water/acetonitrile gradient in the presence of 0.1% trifluoroacetic acid at a flow rate of 20 ml/min. Analytical HPLC was performed on a Vydac® diphenyl column (150×4.6 mm) The purified peptide was characterized by electrospray mass spectrometry. The ESI spectrum showed charge status +6 to +11. The deconvoluted mass was 9419.4 Da (the sequence-predicted mass is 9417 Da).

The purified peptide precursor (20 mg) was dissolved in 40 ml of buffer (pH 7.5) containing 1 N guanidine, 0.2 M HEPES, 1 mM EDTA, 1.5 mM of reduced form glutathione, and 0.75 mM of oxidized form glutathione. The oxidation reaction was monitored by HPLC. After overnight, the reaction was terminated by adding 500 μl of TFA to the solution, and the solution was directly loaded on a Vydac® diphenyl column (22×250 mm, 10-15μ) and purified by RP HPLC using a water/acetonitrile gradient in the presence of 0.1% trifluoroacetic acid at a flow rate of 20 ml/min. Analytical HPLC analysis was the same as described above. The pooled fractions corresponding to the covalent trimer were further analyzed by high resolution mass spectrometry. The ESI spectrum shows charge status +20 to +27. The deconvoluted mass is 28241.6 Da (the sequence-predicted mass is 28248 Da).

### 3. Recombinant SZN51

BL21(DE3)pLysS competent cells (Invitrogen) were transformed with the plasmids encoding the gene for SZN51 according to the manufacturer's directions. The transformed cells were plated on Luria-Bertani (LB) agar with 50 μg/mL ampicillin and 34 μg/mL chloramphenicol and grown overnight at 37° C. Several colonies were picked from the plates, and LB media with antibiotics was inoculated with a single colony and grown overnight at 37° C. with shaking at 225 rpm. Glycerol stocks were also prepared for each colony from the overnight cultures, and the glycerol stocks were used as the starting material for future scale-up expression experiments. Fresh LB media with antibiotics was inoculated with a 1:40 dilution of the overnight preculture, and grown at 37° C. until the optical density at 600 nm reached between 0.6 and 0.8. Protein expression was then induced by addition of 0.5 mM isopropyl-b-D-thiogalactopyranoside (IPTG). The cultures were grown for an additional 3-4 hours at 37° C., and then the cells were harvested by centrifugation at 10,000×g for 15 minutes at 4° C. The cell pellets were stored at −20° C.

The cells were resuspended in lysis buffer and lysed by 3 passes through the microfludizer. The lysate was then clarified by centrifugation. SDS-PAGE and western blot analysis confirmed that the majority of the SZN51 product was detected in the insoluble fraction. Accordingly, washed inclusion bodies were prepared from the insoluble fraction by repeated homogenization and centrifugation steps. The final washed inclusion bodies were pelleted by centrifugation and frozen at −70° C.

Washed inclusion bodies (0.2 g) were dissolved in 20 ml of 15% acetonitrile/water with 0.1% of TFA. After filtering through 0.45 um PVDF disc (Whatman), the solution was directly loaded on a Vydac® C4 column (22×250 mm, 300 Å) and purified by reverse phase HPLC using a water/acetonitrile gradient in the presence of 0.1% trifluoroacetic acid at a flow rate of 15 ml/min. Analytical HPLC was performed on a Vydac® C4 column (150×4.6 mm) The purified peptide was characterized by electrospray mass spectrometry. The ESI spectrum showed a charge status of +5 to +12. The deconvoluted mass was 9249.4 Da (sequence-predicted=9243 Da).

### 4. Recombinant 5-Helix

Frozen recombinant E. coli cells expressing C-terminally histidine-tagged 5-helix peptide were thawed and resuspended in 50 mM Tris-HCl, pH 8.0 and 0.3 M NaCl. A lysate was prepared with a microfluidizer (two passes @˜18,000 psi). The insoluble fraction was collected by centrifugation, and the supernatant was discarded. The insoluble fraction was washed by multiple rounds of resuspension and centrifugation using 50 mM Tris-HCl, pH 8.0, 0.3M NaCl, and 0.05% Triton X-100. The washed insoluble fraction was dissolved in 8 M guanidine hydrochloride (Gd—HCl). The guanidine-soluble extract was mixed with a slurry of IMAC resin and mixed at 65° C. for one hour. The slurry was allowed to cool and the resin allowed to settle by gravity. The cooled resin was transferred to a glass chromatography column and column was washed with 50 mM sodium phosphate, 20 mM imidazole, 0.3 M sodium chloride and 8 M Gd—HCl, pH 8.0. The 5-helix peptide was eluted from the column with 50 mM sodium phosphate, 300 mM imidazole, 0.3 M sodium chloride and 8 M Gd—HCl, pH 8.0. The IMAC Product was spiked with trifluoroacetic acid (TFA) and acetonitrile (ACN) to 0.1% and 5%, respectively, and purified by reverse-phase chromatography at 50° C. The 5-helix peptide was eluted from the column with a linear gradient from 5% to 80% ACN in 0.1% TFA. The peptide-containing fractions were pooled and ACN was removed by evaporation under a stream of nitrogen gas. The 5-helix peptide was further purified by preparative size-exclusion chromatography using a Superdex® 200 column with 50 mM Tris HCl, pH 8.0 and 150 mM NaCl as running buffer. Fractions containing monomeric 5-helix peptide were pooled, sterile-filtered, and snap-frozen in small aliquots in liquid nitrogen for long-term storage at −70° C.

### Peptide Characterization

### 1. Circular Dichroism

All measurements were performed on a J-810 spectropolarimeter (Jasco, Inc., Easton, Md.) at 20° C., using a rectangular quartz cell of 0.1 cm path length. Spectra were acquired using a 1 second time response and a 100 nm/min scan speed and averaged for five acquisitions. Stock solution concentration was determined by quantitative amino acid analysis. Standard measurements were performed on solutions of peptide in sodium acetate (25 to 50 mM), NaCl (50 to 150 mM), pH 4 to 4.5. The percentage of α-helix was calculated based on the molar ellipticity at 222 nm according to Chen et al., 1974, Biochemistry 13:3350-3359. Thermal stability was determined by monitoring the change in the CD signal at 222 nm as a function of temperature, using a 2° C./min increase. The melting temperatures (Tₘ) were determined from the midpoints of the cooperative thermal unfolding transitions. For peptides with Tₘ>90° C., thermal denaturation experiments were also performed in the presence of 2 M guanidine hydrochloride.

### 2. Analytical Ultracentrifugation

All analytical ultracentrifugation (AU) experiments were performed with a XL-I analytical ultracentrifuge (Beckman Coulter, Inc., Indianopolis, Ind.) at 20° C. For sedimentation velocity analysis, the samples were centrifuged at 48,000 rpm at 20° C. for 5 hours, with radial absorbance scans taken approximately every 4 minutes. g*(s) analysis was performed with the program DCDT+, version 2.2.1 (John Philo, Thousand Oaks, Calif.) or the Optima XL-I data analysis software (Beckman Coulter, Inc.). Sedimentation equilibrium experiments were conducted at three different loading concentrations and three different rotor speeds (16,000, 20,000, and 30,000). Molecular masses were calculated using the Optima XL-I data analysis software or using Heteroanalysis version 1.1.33 (from the Analytical Ultracentrifugation Facility, Biotechnology and Bioservices Center of the University of Connecticut).

### 3. D5/5-Helix Competitive Binding Assay (DCBA)

An in vitro binding assay based on fluorescence resonance energy transfer (FRET) was performed as previously described in Caulfield et al., 2010, J Biol Chem 285:40604-40611. Briefly, the assay uses D5 IgG (see U.S. Pat. No. 7,744,887) conjugated to europium cryptate (Eu-D5) and a biotinylated derivative of the recombinant gp41 mimetic 5-helix (5H). Biotin-5H binds to a streptavidin-conjugated allophycocyanin (APC) substrate to form a 5H-SA-APC complex. Binding of Eu-D5 to 5H results in FRET from Eu to APC. When the reaction system is excited at a wavelength of 340 nm, the amount of bound Eu-D5 is measured at the emission wavelength of 665 nm, and total Eu is measured at the emission wavelength of 620 nm. Data are reported as 10000× the ratio of signal at 665 nm to signal at 620 nm. Agents that bind competitively to either component cause a decrease in the ratio value.

4. p4-2R5 Neutralization Assay

This neutralization assay was performed as previously described in Joyce et al., 2002, J Biol Chem 277:45811-45820 with the following modifications: HeLa P4R5 cells were seeded at 1000 cells/well in a 384-well plate and infected the following day with the appropriate HIV-1 or SHIV virus at a multiplicity of infection of approximately 0.01 in the presence of serial dilutions of immune sera or fractionated IgG 48 hours post infection, and cells were lysed and β-galactosidase activity was measured using a chemiluminescent substrate (GalScreen®, Applied Biosystems™, Life Technologies Corp., Carlsbad, Calif.). For analysis of purified IgG, the data are expressed as IC₅₀, defined as the IgG concentration resulting in 50% reduction of chemiluminescence signal. For analysis of sera, the IC₅₀ is defined as the reciprocal serum dilution resulting in 50% reduced chemiluminescence signal.

### Results

Peptide characterization consisted of biophysical assessment of secondary structure by CD spectroscopy and oligomeric state by AUC. Integrity and presentation of the D5 epitope was evaluated in the DCBA and P4/2R5 neutralization assay to measure the proper presentation of the hydrophobic pocket contained within the pre-hairpin intermediate.

Table 2 summarizes the data for the peptide constructs produced. In general, solubility of peptides was optimal in the pH range of 3 to 5, so CD and AUC determinations performed in either water where the TFA counter-ion resulted in a pH of ˜3.5 or in sodium acetate buffer, pH 4 were most reliable. In this pH range both monomeric and trimeric peptide constructs showed a high percentage of α-helical structure, consistent with prediction. NHR-based peptide constructs showed an increasing tendency toward aggregation and precipitation as the pH was increased, particularly in the range from pH 6 to 8. The monomeric N51, N51-2B, and N51-3B peptides all exhibit a strong preference for self-association as evidenced from the AUC ratio of observed to predicted molecular weight. However, most of these constructs were relatively unstable at near-neutral pH and exhibited significant aggregation. Early attempts to stabilize N51 by addition of the SZ trimerization domain were partially successful, and in-solution this peptide self-assembled to form an apparent trimer-tetramer equilibrium. Helicity of these peptides was optimal in the full length N51 context as the C-terminal truncated Δ23 versions of both N51 and SZN51 showed reduced helical content. AUC analysis of the N51 and SZN51 family of peptides by sedimentation equilibrium at multiple concentrations and multiple rotor speeds yielded calculated molecular weight values that varied considerably, suggesting that the analysis model did not accurately fit all of the data. The most likely explanation is that while these peptides showed a propensity for self-association, this oligomerization was uncontrolled and multiple forms of increasingly higher order oligomers and aggregates formed over time. By contrast, trimeric N51 peptides stabilized either via oxidation of engineered disulfides (CCIZN51)₃ or by chemical scaffolding such as KTA(N51)₃ showed a very high degree of secondary structure, and AUC have ratios near unity, implying that there is little apparent aggregation or self-association of the trimers.

The ability of the various peptide constructs to present a conformationally correct binding epitope for the neutralizing monoclonal antibody D5 was assessed by measuring their ability to compete for binding to 5-helix in the DCBA assay. IC50 values were comparable across the various constructs with the exception that the mutations used in N51-2B and N51-3B have an apparent negative impact on D5 binding. Although none of the amino acid substitutions in these peptides change the critical D5 contact residues defined as L568, W571, G572, and K574, the common mutation L565A is in close proximity to L568 and may have an unpredicted effect on binding. In contrast, these peptides show comparable IC50 values for viral entry inhibition as native N51, suggesting that the mutations do not adversely affect the ability of the hydrophobic pocket to bind the C-heptad repeat peptides and thus function as a dominant negative inhibitor of fusion. In general, the relatively constant antiviral activity observed across the peptide series provides qualitative evidence that proper presentation of the pre-hairpin intermediate structure is maintained. It is of note that an approximate 10-fold enhancement of inhibitory potency for V570A is realized in KTA(N51)₃.

TABLE 2
Biophysical and functional assessment of purified peptide constructs

| | | | | | | | | | p4/2R5 | |
|---|---|---|---|---|---|---|---|---|---|---|
| | CD | | | AUC | | | | DCBA | IC₅₀ (nM) | |
| Peptide | % (-helix | Condition | Tm (° C.) | Predicted Mw | Observed Mw | Ratio | Condition | IC₅₀ (nM) | V570A | HXB2 |
| N51 (L′587) | 95 | b | NA | 5936 | 15490 | 2.6 | a | 110 | 20.2 | 104 |
| KTA(N51)3 | 98 | b | NA | 18533 | 25200 | 1.4 | a | 88.3 | 1.49 | 151 |
| (L′990) | | | | | | | | | | |
| r(CCIZN51)3 | 95 | b | NA | 28250 | ppt (pH 7) | ND | NA | 0.997 | 23.2 | 71.2 |
| (L′316) | | | | | | | | | | |
| ChA(N51)3 | NA | | NA | 19306 | NA | NA | NA | 92.8 | 56.4 | 194 |
| N51-2B (L′583) | 97 | a | NA | 6036 | 22385 | 3.6 | a | >300 | 33.4 | 60.9 |
| N51-3B (L′584) | 105 | a | NA | 5936 | 20104 | 3.3 | a | >300 | 49.3 | 107 |
| KTA(N51-3B)3 | 88 | c | NA | 18407 | ppt (pH 7) | ND | NA | 38.4 | 29.1 | 79.4 |
| (L′510) | | | | | | | | | | |
| rSZN51 (L′3651) | 129 | e | NA | 9244 | 34862 | 3.8 | c | | 52.1 | 10.13 |
| rSZN51Δ23 | 139 | e | NA | 6520 | 20755 | 3.2 | c | | | 1514.4 |
| SZN51Δ23 | 72 | f | NA | 6389 | NA | | | | | |
| N51Δ23 | 10 | f | NA | 3212 | NA | | | | | |
| (CCSZN51 )3 | 90 | a | NA | 29118 | 39952 | 1.4 | b | ND | ND | ND |
| (L′928) | | | | | | | | | | |
| Recomb. 5-helix | 75 | | >90 | 25397 | 25014 | 0.98 | | 1002 | 0.82 | 12.4 |
| CONTROL D5 | | | | | | | | 0.088 | 61.7 | 179 |
| CONTROL T20 | | | | | | | | NA | 8.66 | 8.25 |

CD Condition:
a: 50 mM Na Acetate, 0.15M NaCl, pH 4
b: water
c: 25 mM Na Acetate, pH 4
d: 25 mM Na Acetate, 0.05M NaCl, pH 4
e: 10 mM sodium phosphate, 0.05M NaCl, pH 7
f: 10 mM sodium phoshpate, pH 7
AUC Condition:
a: 25 mM Na Acetate, 0.15M NaCl, pH 4
b: 25 mM Na Acetate, 0.05M NaCl, pH 4
c: 50 mM HEPES, 0.05M NaCl, pH 7.3

### Example 2

### Serology

### 1. ELISA

Serum end point dilutions were determined by testing immune serum samples against biotinylated peptide (CCIZN 17)₃ added directly to streptavidin coated 96-well plates (Thermo Fisher Scientific, Inc., Pittsburgh, Pa.). The biotinylated peptide was coated at a concentration of 4 μg/ml in PBS per well, overnight at 4° C. Plates were washed six times with PBS containing 0.05% Tween-20 (PBST) and blocked with PBST containing 3% (v/v) non-fat dry milk (PBST-milk). Testing samples, pre immune and immune samples were diluted, starting at 1:100 and serial diluted 4 fold, eight times in a final volume of 100 μl per well. Plates were incubated for 2 hours at room temperature, followed by six washes with PBST. Fifty microliters of either HRP-conjugated goat anti-guinea pig (Jackson ImmunoResearch Laboratories, Inc., West Grove, Pa.) or goat anti-human (Invitrogen) secondary antibodies were diluted in PBST-milk at either 1:5000 or 1:2000, receptively and added to each well and incubated for one hour at room temperature. Plates were washed six times, followed by the addition of substrate (TMB; Virolabs, Inc., Chantilly, Va.) in 100 μl per well and stopped with TMB-stop solution after 3-5 minutes of development. The antibody titer was determined as the reciprocal of the highest dilution that gave an OD at 450 nm value above the mean plus 2 standard deviations of the conjugate control wells.

### 2. D5/5-Helix Competitive Binding Assay (DCBA)

An in vitro binding assay based on fluorescence resonance energy transfer (FRET) was performed as previously described in Caulfield et al., 2010, J Biol Chem 285:40604-40611. Briefly, the assay uses D5 IgG conjugated to europium cryptate (Eu-D5) and a biotinylated derivative of the recombinant gp41 mimetic 5-helix (5H). Biotin-5H binds to a streptavidin-conjugated allophycocyanin (APC) substrate to form a 5H-SA-APC complex. Binding of Eu-D5 to 5H results in FRET from Eu to APC. When the reaction system is excited at a wavelength of 340 nm, the amount of bound Eu-D5 is measured at the emission wavelength of 665 nm, and total Eu is measured at the emission wavelength of 620 nm. Data are reported as 10000× the ratio of signal at 665 nm to signal at 620 nm. Agents that bind competitively to either component cause a decrease in the ratio value.

### 3. Neutralization Assays

a. P4/2R5 Assay

Assay performed as previously described in Joyce et al., 2002, J Biol Chem 277:45811-45820 with the following modifications: HeLa P4R5 cells were seeded at 1000 cells/well in a 384-well plate and infected the following day with the appropriate HIV-1 or SHIV virus at a multiplicity of infection of approximately 0.01 in the presence of serial dilutions of immune sera or fractionated IgG. 48 hours post infection, cells were lysed and β-galactosidase activity was measured using a chemiluminescent substrate (GalScreen, Applied Biosystems). For analysis of purified IgG, the data are expressed as IC₅₀, defined as the IgG concentration resulting in 50% reduction of chemiluminescence signal. For analysis of sera, the IC₅₀ is defined as the reciprocal serum dilution resulting in 50% reduced chemiluminescence signal.

b. TZM-bl Assay

Neutralization was measured as a reduction in luciferase reporter gene expression after a single round of infection in TZM-bl cells as previously described. See Montefiori (2004) in Current Protocols in Immunology eds Coligan et al. (John Wiley & Sons) Dec. 11, 2001-Dec. 11, 2015 and Li et al., 2005, J Virol 79:10108-10125. TZM-bl cells were obtained from the NIH AIDS Research and Reference Reagent Program, as contributed by John Kappes and Xiaoyun Wu. Briefly, 200 TCID₅₀ of virus was incubated with serial 3-fold dilutions of test sample in duplicate in a total volume of 150 μl for 1 hour at 37° C. in 96-well flat-bottom culture plates. Freshly trypsinized cells (10,000 cells in 100 μl of growth medium containing 75 μg/ml DEAE dextran) were added to each well. One set of control wells received cells and virus (virus control) and another set received cells only (background control). After a 48-hour incubation, 100 μl of cells was transferred to a 96-well black solid plate (Costar®) for measurements of luminescence using the Britelite Luminescence Reporter Gene Assay System (PerkinElmer Inc., Waltham, Mass.). Neutralization titers are the dilution at which relative luminescence units (RLU) were reduced by 50% compared to virus control wells after subtraction of background RLUs. Assay stocks of molecularly cloned Env-pseudotyped viruses were prepared by transfection in 293T cells and were titrated in TZM-bl cells as described in and Li et al., 2005, J Virol 79:10108-10125. Clade A, B and C reference Env clones have been described previously. See Li et al., 2005, J Virol 79:10108-10125; Li et al., 2006, J Virol 80:11776-11790; and Blish et al., 2007, AIDS 21:693-702.

c. A3R5 Assay

The assay measures neutralization in 96-well microdilution plates as a function of a reduction in luciferase reporter gene expression. A3R5 cells (A3.01/R5.6) were provided by the US Medical HIV Research Program (MHRP). This is a derivative of the human lymphoblastoid cell line, CEM, that naturally expresses CD4 and CXCR4 and was engineered at MHRP to express CCR5. See Folks et al., 1985, Proc. Nall. Acad. Sci. (USA) 82:4539-4543. The cells were moderately permissive to infection by most strains of HIV-1. DEAE dextran was used in the medium during neutralization assays to enhance infectivity. Because the cell line does not contain a reporter gene, molecularly cloned viruses must be used that carry a reporter gene in the viral genome. Env-expressing infectious molecular clones carrying a Renilla Luciferase reporter gene (Env.IMC.LucR viruses) provide suitable infection for neutralization assays. Expression of the reporter genes was induced in trans by viral Tat protein soon after infection. Luciferase activity was quantified by luminescence and is directly proportional to the number of infectious virus particles present in the initial inoculum. The assay was performed in 96-well culture plates for high throughput capacity. Use of a clonal cell population provided enhanced precision and uniformity. The assay has been standardized for multiple rounds of infection with Env.IMC.LucR viruses produced by transfection in 293T cells.

### Example 3

### HIV 350 and 365: Guinea Pig Immunogenicity

Duncan-Hartley guinea pigs (HIV-350, n=8 per group*) were immunized, intramuscularly with 100 micrograms of peptide immunogen three times at weeks 0, 4, and 8. Peptides, reconstituted in 20 mM Hepes buffer, neutral pH, were formulated in 180 μg of aluminum hydroxyphosphate sulfate (Merck & Co., Inc.) plus 40 μg of Iscomatrix Adjuvant™ (CSL, Inc.) per dose. Serum samples were collected via whole blood in serum separator tubes at weeks 7 and 11 for each animal as well as several serum collections prior the first immunization (pre-bleed).

Study HIV-350 had tested the peptide construct SZN51. Table 3a shows the immunization protocol for this group in the study.

Duncan-Hartley guinea pigs (HIV-365, n=6 per group) were immunized intramuscularly with 30 μg of peptide immunogen three times at weeks 0, 4, and 8. Peptides, reconstituted in 20 mM HEPES buffer, neutral pH, were formulated in 180 μg of aluminum hydroxyphosphate sulfate (Merck & Co., Inc.) plus 40 μg of Iscomatrix Adjuvant™ (CSL, Inc.) per dose. Serum samples were collected via whole blood in serum separator tubes at weeks 3, 7 and 11 for each animal. Serology was performed as described in Example 2.

Study HIV-365 assessed (1) a series of constrained and stabilized N51 trimeric peptides consisting of synthetic KTA(N51)₃, KTA(N51-2B)₃, KTA(N51-3B)₃, ChA(N51)₃, and recombinant (CCIZN51)₃; and (2) homologous (CCIZN36)₃ immunization compared with a regimen of (CCIZN36)₃ followed by KTA(N51)₃ and 5-Helix. Table 3b shows the immunization protocol and group designation for the study. Serology was performed using the p4/2R5 neutralization assay and using the TZM-bl assay (virus V570A) and the A3R5 assay.

TABLE 3a
Immunization schedule for HIV-350

| Group | Dose 1 | Dose 2 | Dose 3 |
|---|---|---|---|
| 350-8 | SZN51 | SZN51 | SZN51 |

TABLE 3a
Immunization schedule for HIV-365

| Group | Dose 1 | Dose 2 | Dose 3 |
|---|---|---|---|
| 365-1 | (CCIZN51)₃ | (CCIZN51)₃ | (CCIZN51)₃ |
| 365-2 | KTA(N51)₃ | KTA(N51)₃ | KTA(N51)₃ |
| 365-3 | KTA(N51-2B)₃ | KTA(N51-2B)₃ | KTA(N51-2B)₃ |
| 365-4 | KTA(N51-3B)₃ | KTA(N51-3B)₃ | KTA(N51-3B)₃ |
| 365-5 | ChA(N51)₃ | ChA(N51)₃ | ChA(N51)₃ |

### Results

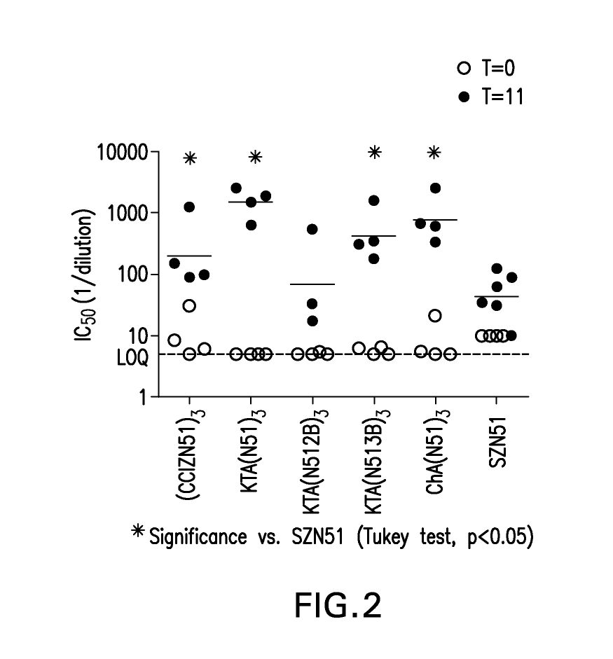

FIG. 2 presents a summary of the neutralization assay data for the N51 peptide series as assayed against virus V570A in the p4/2R5 assay. The non-covalently constrained recombinant SZN51 peptide is demonstrably inferior to all of the remaining peptide immunogens in terms of its ability to elicit a neutralizing antibody response. This clearly demonstrates that covalent stabilization of N-peptides achieved by scaffolding with either CCIZ or chemical scaffold cores is critical to eliciting the desired functional immune response.

Table 4 presents a summary of neutralizing antibody titers as determined at T=11 time point, 3 weeks after the final dose for both individual animals and as the calculated geomean. Uniformly, groups containing an N51 peptide in combination with either (CCIZN36)₃ or 5-helix were superior to (CCIZN36)₃ alone with respect to elicitation of higher neutralizing antibody titers across all viral strains tested.

TABLE 4
Serology summary for Guinea pig study HIV-365

| | VERTICAL and | | | | | | | |
|---|---|---|---|---|---|---|---|---|
| | TZM-bl IC₅₀ (1/diI) | | | A3R5 IC₅₀ (1/diI) | | | | |
| | V570A | | HXB2 | 9020.A13 | SC22.3C2 | RHPA | MW965.26 | Ce0393_C3 |
| Animal | M | D | T = 11 | T = 11 | T = 11 | T = 11 | T = 11 | T = 11 |
| Group 7: 30 mcg (ccIZN36)3 1X | | | | | | | | |
| MAA + 40 mcg IMX/dose | | | | | | | | |
| 1 | 255 | 288 | 10.2 | 187 | 23.0 | 21 | 20.0 | 151 |
| 2 | 138 | 60 | 7.3 | 66 | 41.0 | 25 | 20.0 | 59 |
| 3 | 1014 | 412 | 5.1 | 233 | 41.0 | 29 | 20.0 | 170 |
| 4 | 94.1 | 293 | 5.8 | 157 | 24.0 | 20 | 25.0 | 161 |
| 5 | 2557 | 103 | 11.9 | 60 | 28.0 | 20 | 20.0 | 72 |
| 6 | 2299 | 103 | 5.7 | 87 | 30.0 | 20 | 20.0 | 54 |
| GeoMean | 520 | 168 | 7 | 115 | 30 | 22 | 21 | 99 |
| Group 8: 30 mcg | | | | | | | | |
| (ccIZN36)3/KTA(N51)3/5-Helix | | | | | | | | |
| (PRIME BOOST) 1X MAA + | | | | | | | | |
| 40 mcg IMX/dose | | | | | | | | |
| 1 | 2557 | 924 | 10.2 | 539 | 71.0 | 39 | 46.0 | 376 |
| 2 | 2557 | 527 | 6.2 | 407 | 94.0 | 53 | 61.0 | 371 |
| 3 | 2420 | 478 | 49.4 | 221 | 59.0 | 45 | 36.0 | 204 |
| 4 | 2557 | 512 | 8.0 | 211 | 56.0 | 33 | 30.0 | 128 |
| 5 | 2009 | 1574 | 6.3 | 281 | 62.0 | 38 | 47.0 | 234 |
| 6 | 1130 | 260 | 6.3 | 257 | 45.0 | 37 | 40.0 | 165 |
| GeoMean | 2124 | 604 | 10 | 301 | 63 | 40 | 42 | 228 |

### Example 4

### HIV 360: Non-Human Primate Immunogenicity

This NHP study was conducted in two phases to (1) test dosing effect of multiple (CCIZN36)₃ immunizations and (2) assess benefit of heterologous antigen administration in animals primed with (CCIZN36)₃.

Rhesus macaques (Macaca mulatta), three per group, were immunized at 0, 1 and 2, months with either 100, 300, or 1000 μg (CCIZN36)₃. At 34 weeks, all groups were boosted with an additional dose of 100 micrograms (CCIZN36)₃. Seven months after the boost immunization, the monkeys were regrouped so that all groups had equal representation of all doses of (CCIZN36)₃. Each group was immunized with the following protocols with 4-week intervals: Group 1 was given two immunizations of (CCIZN36)₃; Group 2 was immunized sequentially with KTA(N51)₃ and 5-helix; and Group 3 was immunized sequentially with 5-helix and KTA(N51)₃. Peptides, reconstituted in 20 mM HEPES buffer, neutral pH or 5-Helix were formulated in 180 μg of aluminum hydroxyphosphate sulfate (Merck & Co., Inc.) plus 40 μg of Iscomatrix Adjuvant™ (CSL, Inc.) per dose. Whole blood for serum at indicated time points were collected and used for serology analysis, including binding and viral neutralization assays.

Table 5 summarizes the protocol for the complete study.

TABLE 5
Immunization protocol for Non-Human Primate study HIV-360

| Grp # | T = 0 | T = 4 | T = 8 | T = 34 | T = 62 | T = 66 |
|---|---|---|---|---|---|---|
| 1 | 0.1 mg | 0.1 mg | 0.1 mg | 0.1 mg | 0.1 mg | 0.1 mg |
| | (CCIZN36)₃ | (CCIZN36)₃ | (CCIZN36)₃ | (CCIZN36)₃ | (CCIZN36)₃ | (CCIZN36)₃ |
| 2 | 0.3 mg | 0.3 mg | 0.3 mg | 0.1 mg | 0.1 mg | 0.1 mg |
| | (CCIZN36)₃ | (CCIZN36)₃ | (CCIZN36)₃ | (CCIZN36)₃ | KTA(N51)₃ | 5-Helix |
| 3 | 1 mg | 1 mg | 1 mg | 0.1 mg | 0.1 mg | 0.1 mg |
| | (CCIZN36)₃ | (CCIZN36)₃ | (CCIZN36)₃ | (CCIZN36)₃ | 5-Helix | KTA(N51)₃ |

Phase 2 (comparative homologous vs. heterologous antigen administration occurred at weeks 62 through 66.

### Results

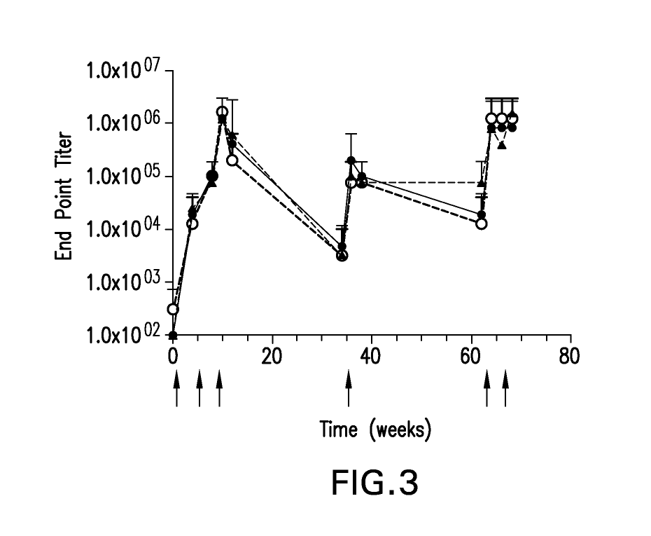

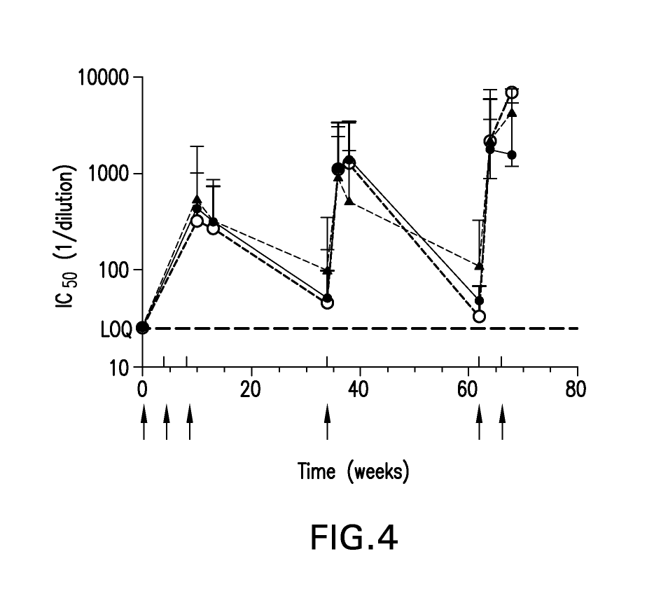

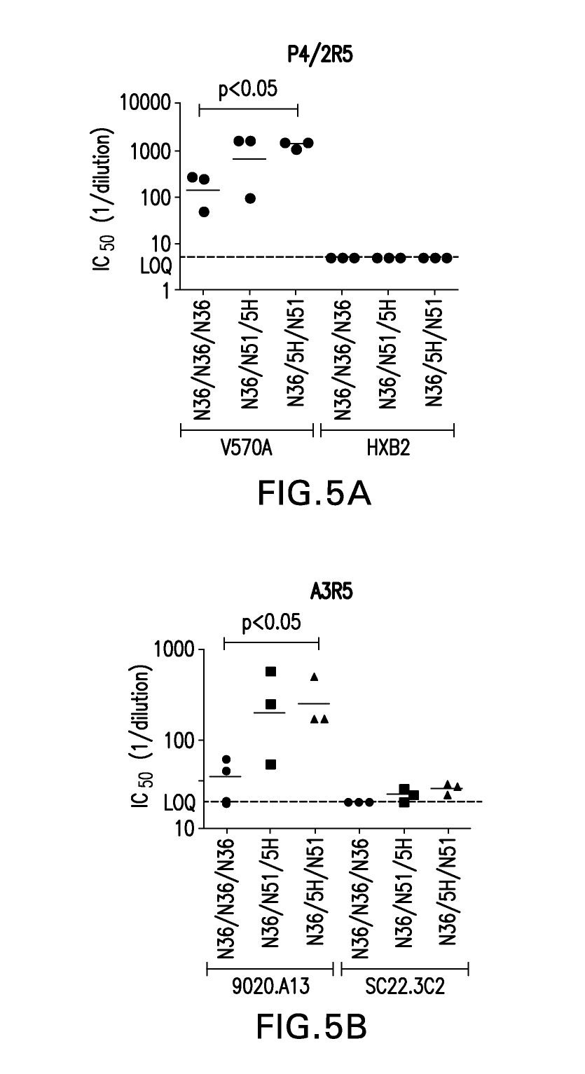

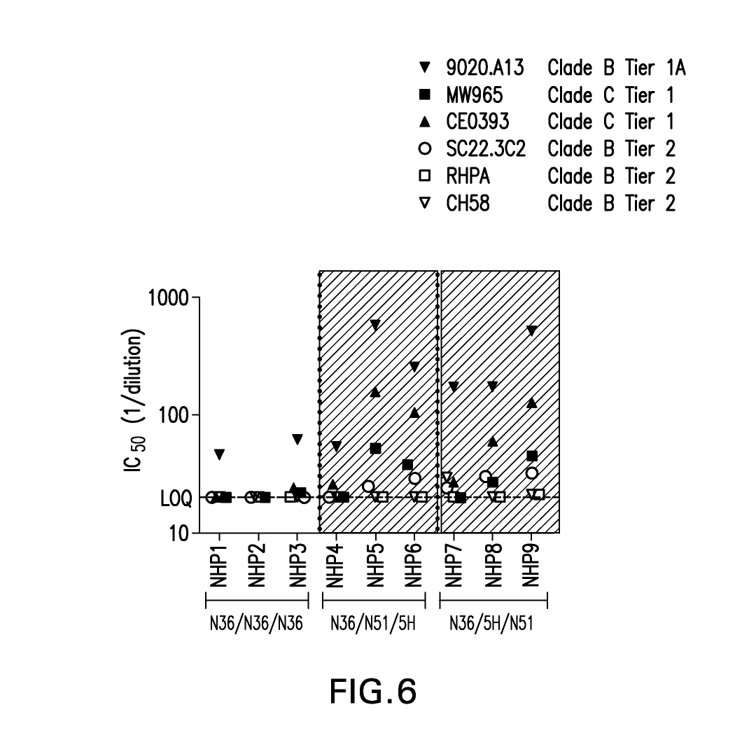

FIGS. 3 and 4 summarize the ELISA and DCBA assay results for the whole study. ELISA and DCBA at week 11 (3 weeks post-dose 3) had shown good responses, but neutralizing antibody titers in the p4/2R5 assay were low even against the D5 hypersensitive V570A_HXB2 virus (data not shown). All animals were then administered a fourth dose of 100 μg of (CCIZN36)₃ at 34 weeks. Both ELISA and DCBA titers declined after this 26 week rest period and both were boosted as analysis of the T=36 week bleed showed. Although ELISA titers did not reach previous peak levels DCBA titer, a measure of functional D5-like specificity, reached higher levels. However, once again the p4/2R5 neutralizing antibody titers were lower than would have been predicted from previous guinea pig experiments where immunized animals developed comparable ELISA and DCBA responses. At this point, the animals in each group were shuffled to eliminate any possible bias, and following another rest period, the heterologous antigens were used in sequential combination for two of the three study groups. In this case, Group 1 received two additional doses of (CCIZN36)₃ while Group 2 received KTA(N51)₃ followed by 5-helix and Group 3 received 5-helix followed by KTA(N51)₃. ELISA and DCBA titers had significantly declined during the interim rest period between 34 and 62 weeks but were boosted upon immunization. Important differences in neutralization titers were clearly observed in groups which received heterologous antigens as is apparent from FIGS. 5A-B and 6. Both the magnitude and breadth of neutralizing antibody responses measured in P4/2R5 and A3R5 assays is significantly enhanced in Groups 2 and 3.

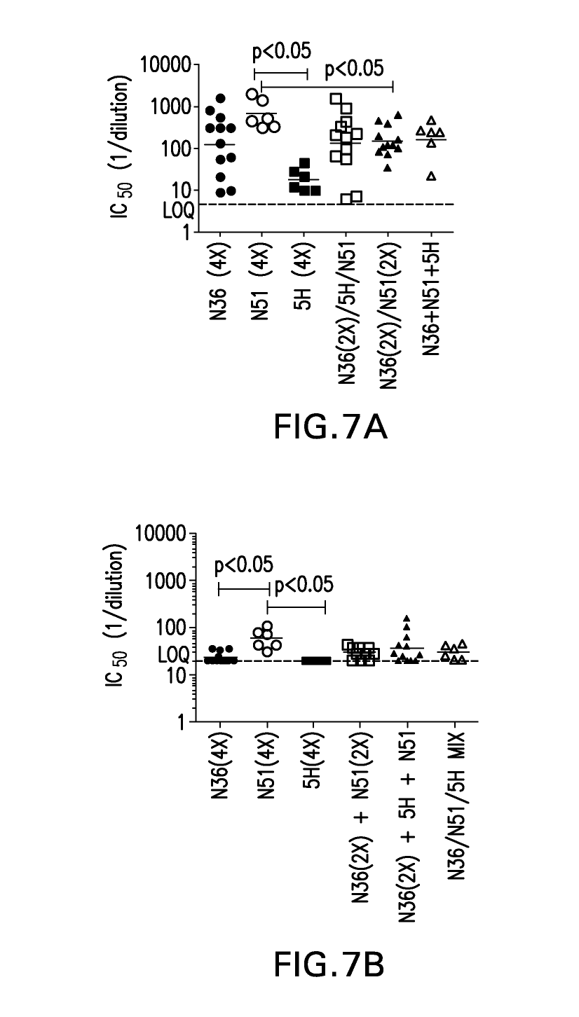

FIGS. 7A-B presents a comparison of P4/2R5 neutralizing antibody titers determined during Phase 1 and Phase 2 of the study. Responses against V570A_HXB2 were not measured during either phase, but responses against V570A are clearly enhanced in the heterologous groups in terms of number of animals responding and magnitude of the neutralizing antibody titers.

### Example 5

### HIV 366: Non-Human Primate Immunogenicity

The purpose of this NHP study was to expand upon the results of study HIV-360 and to determine whether the enhancement observed in the heterologous groups was attributable to the addition of KTA(N51)₃ or 5-helix or whether both were required.

Rhesus macaques (Macaca mulatta), six per group, were immunized with 100 μg per dose of formulated antigen. One group received a series of four homologous immunizations of an equal mixture of all three tested antigens at 100 μg per antigen. The immunizations were given at 0, 1, 6 and 8 months. Peptides, reconstituted in 20 mM HEPES buffer, neutral pH or 5-Helix were formulated in 180 μg of aluminum hydroxyphosphate sulfate (Merck & Co., Inc.) plus 40 μg of Iscomatrix Adjuvant™ (CSL, Inc.) per dose. Whole blood for serum at indicated time points were collected and used for serology analysis, including binding and viral neutralization assays.

Table 6 summarizes the protocol for the complete study. Groups 1 and 5 contain identical antigen combinations to those tested in HIV-360. In addition, the efficacy of homologous administration of KTA(N51)₃ or 5-helix (Groups 2 and 3) was tested. Group 4 tested the efficacy of (CCIZN36)₃ and KTA(N51)₃ administered sequentially while group 6 tested a multiple antigen combination in which all immunogens were administered concurrently at each dose.

TABLE 6
Immunization protocol for Non-Human Primate study HIV-366

| Grp # | T = 0 | T = 4 | T = 24 | T = 36 |
|---|---|---|---|---|
| 1 | (ccIZN36)₃ | (ccIZN36)₃ | (ccIZN36)₃ | (ccIZN36)₃ |
| 2 | KTA(N51)₃ | KTA(N51)₃ | KTA(N51)₃ | KTA(N51)₃ |
| 3 | 5-Helix | 5-Helix | 5-Helix | 5-Helix |
| 4 | (ccIZN36)₃ | (ccIZN36)₃ | KTA(N51)₃ | KTA(N51)₃ |
| 5 | (ccIZN36)₃ | (ccIZN36)₃ | 5-Helix | KTA(N51)₃ |

### Results

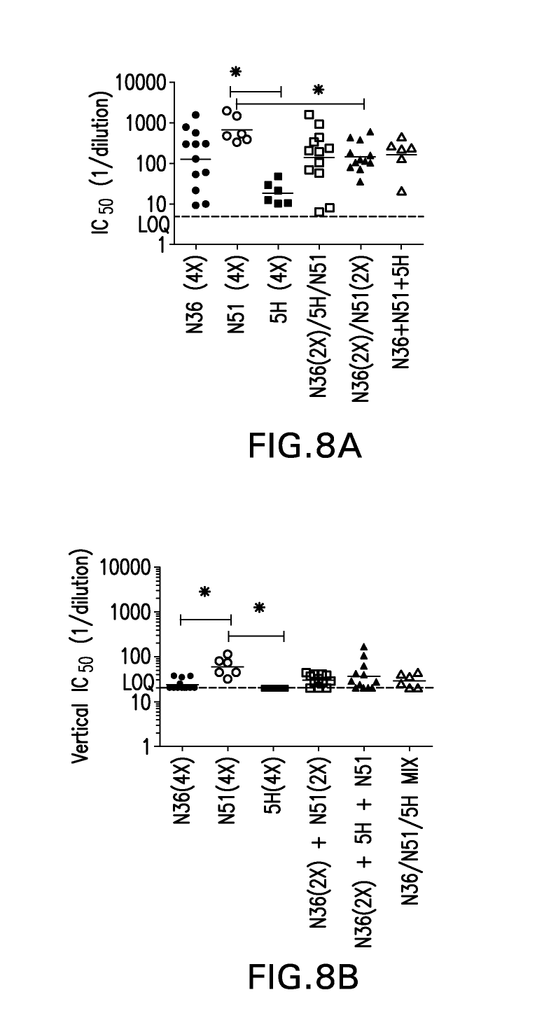

FIGS. 8A-B show the neutralizing antibody titers against virus V570A_HXB2 determined in the P4/2R5 and TZM-bl assays at the T=38 week bleed collected two weeks after the final immunization. The most potent neutralization titers in both assays are achieved by homologous immunization with KTA(N51)₃ alone, although groups which contain this peptide in combination with either (CCIZN36)₃ or both (CCIZN36)₃ and 5-helix trend toward better potency than (CCIZN36)₃ alone. The combined mixture of three immunogens does not appear to significantly differ from sequential administration.

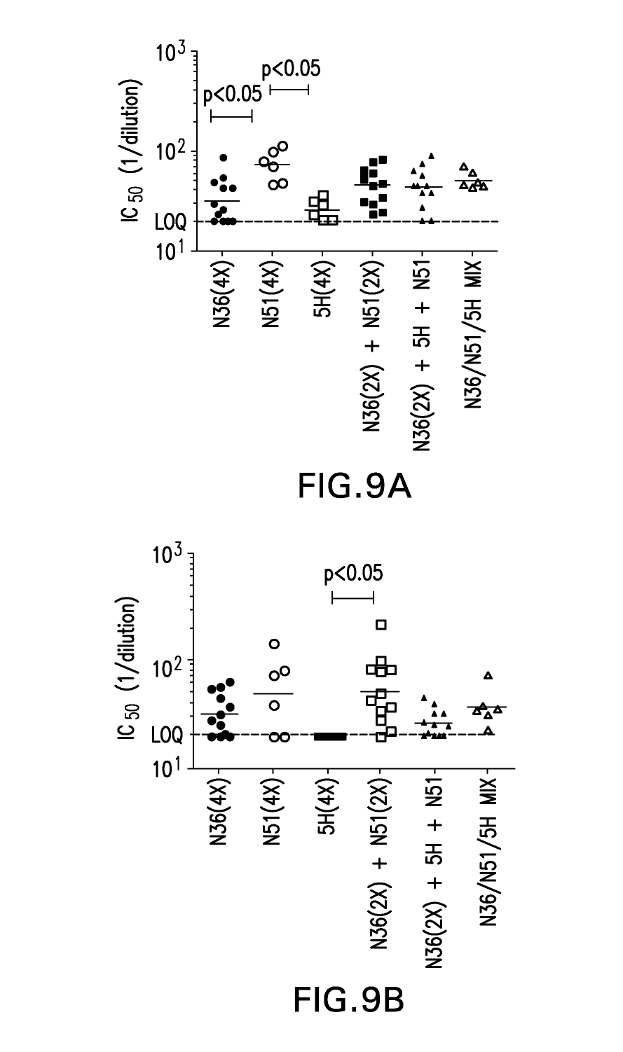

The positive neutralization results for V570A_HXB2 were confirmed independently in the A3R5 assay using two Tier 1 clade C viral isolates, Ce0393 and MW965, as shown in FIGS. 9A-B. Results for Ce0393 were generated using the two week post dose 4 bleed at 38 weeks and for Mw965 using the two week post dose 3 bleed at 26 weeks. As was observed for the V570A_HXB2 hypersensitive isolate, the most potent neutralization titers were observed in the homologous KTA(N51)₃ group while other groups containing this peptide trended toward higher titers relative to (CCIZN36)₃ or 5-helix alone. Indeed, in all assays, 5-helix alone was extremely poor at eliciting neutralizing antibody titers although it was potent immunologically as determined by ELISA (data not shown). Furthermore, this analysis provides evidence for the ability of these immunogens to induce cross-clade protection since the data shows positive neutralization of two clade C viruses using peptides whose sequence is derived from a clade B viral isolate (HXB2).

Table 7 presents a summary of the geomean of neutralization titers for all groups at various bleeds collected throughout the study. As expected, neutralization titers declined significantly at week 36 at the end of the 12 week rest between doses 3 and 4. For all other time points the KTA(N51)₃ group displayed the highest neutralization titers relative to homologous (CCIZN36)₃ or 5-helix (Groups 2 and 3) while groups containing N51 also trended higher than homologous (CCIZN36)₃ or 5-helix.

TABLE 7
Comparison of neutralizing antibody geomean titers for immunogen regimens at
various timepoints in NHP study HIV-366.

| | Neutralizing Antibody IC₅₀ (1/dilution) | | | | | | | |
|---|---|---|---|---|---|---|---|---|
| | p4/2R5 | | TZM-bl | ZM-bl(FcgR) | A3R5 | | | |
| | V570A | V570A | V570A | MW965 | Ce0390 | 9020.A13 | SC22.3C2 | CH58 |
| Group | HIV-366 | HIV-360 | HIV-360 | HIV-366 | HIV-366 | HIV-360 | HIV-360 | HIV-366 |
| (ccIZN36)3 | 36 | 91 | 24 | 32 | 26 | 53 | <20 | 20 |
| (ccIZN36)₃ + | 114 | 308 | ND | 53 | 45 | ND | ND | 22 |
| KTA(N51)₃ | | | | | | | | |
| (ccIZN36)₃ + 5-Helix | 28 | 152 | ND | 26 | 29 | ND | ND | 21 |
| (ccIZN36)₃+ | NA | 675 | 105 | NA | NA | 201 | 27 | NA |
| KTA(N51)₃ + 5-Helix | | | | | | | | |
| (ccIZN36)₃ + 5-Helix + | NA | 1348 | 87 | NA | NA | 250 | 28 | NA |
| KTA(N51)₃ | | | | | | | | |

NA: Data not available
ND: Not determined

In aggregate, the results from study HIV-366 provides strong support for the hypothesis that the enhanced neutralization potency observed in study HIV-360 is attributable to the addition of KTA(N51)₃ and not 5-helix and directly supports the claim that constrained trimeric N51-based N-peptides are superior from a functional immunological perspective.

What is claimed is:

1. A gp41 peptide mimetic comprising a scaffold core which is linked to three N-peptides wherein each N-peptide comprises an amino acid sequence comprising N36 (SGIVQQQNNLLRAIEAQQHLLQLTVWGIKQLQARIL; SEQ ID NO:1) or a modified version thereof, wherein the three N-peptides interact with each other to form a trimeric coiled-coil which mimics the pre-hairpin conformation of HIV gp41, with the proviso that the gp41 peptide mimetic is not (CCIZN36)₃.

2. The gp41 peptide mimetic of claim 1, wherein each of the three peptides is covalently linked to the scaffold core at a different point of attachment.

3. The gp41 peptide mimetic of claim 1, wherein the scaffold core comprises, or consists of, tris(2-carboxyethyl)phosphine hydrochloride; tris-succinimidyl aminotriacetate; tris-(2-maleimidoethyl)amine; KTA-bromide or cholic acid.

4. The gp41 peptide mimetic of claim 3, wherein the scaffold core is KTA-bromide or cholic acid.

5. The gp41 peptide mimetic of claim 1, wherein the scaffold core is a linear polypeptide chain comprising three functionalized residues allowing attachment of three N-peptides.

6. The gp41 peptide mimetic of claim 5, wherein the scaffold core comprises:
(SEQ ID NO: 41)
a) CH₃CO-Ava-Lys-Ava-Lys-Ava-Lys-Ava-NH₂
(SEQ ID NO: 42)
b) CH₃CO-Arg-Lys-Arg-Lys-Arg-Lys-Arg-NH₂;
(SEQ ID NO: 43)
c) CH₃CO-Glu-Lys-Glu-Lys-Glu-Lys-Glu-NH₂;
(SEQ ID NO: 44)
d) CH₃CO-Cys-Arg-Lys-Arg-Lys-Arg-Lys-Arg-NH₂;
or
(SEQ ID NO: 45)
e) CH₃CO-Cys-Glu-Lys-Glu-Lys-Glu-Lys-Glu-NH₂.

7. The gp41 peptide mimetic of claim 2, wherein the scaffold core is a carbocyclic scaffold comprising cyclohexane, cycloheptane or cyclooctane.

8. The gp41 peptide mimetic of claim 2, wherein the scaffold core is a heterocyclic scaffold comprising pyrrolidine, oxolane, thiolane, piperidine, oxane, thiane, azepane, oxepane, thiepane, piperazine, morpholine, or thiomorpholine.

9. The gp41 peptide mimetic of claim 1, wherein one or more N-peptides comprises N51
(SEQ ID NO: 4
(QARQLLSGIVQQQNNLLRAIEAQQHLLQLTVWGIKQLQARILAVERYLK
DQ; N51-2B
(SEQ ID NO: 8)
(QIRELISKIVEQINNILRAIEAQQHALQLTVWGIKQLQARILAVERYLK
DQ
or
N51-3B
(SEQ ID NO: 9)
(QARQLLSGIVQQQNNLLRAIEAQQHALQLTVWGIKQLQARILAVERYLK
DQ.

10. The gp41 peptide mimetic of claim 1, wherein each N-peptide consists of N51
(SEQ ID NO: 4)
(QARQLLSGIVQQQNNLLRAIEAQQHLLQLTVWGIKQLQARILAVERYLK
DQ.

11. The gp41 peptide mimetic of claim 1, wherein the N-peptides comprise the same or different amino acid sequences.

12. The gp41 peptide mimetic of claim 1, wherein the N-peptides consist of the same amino acid sequence.

13. The gp41 peptide mimetic of claim 1, wherein one or more N-peptides are chimeric N-peptides which comprise:
a) a scaffold portion comprising a soluble α-helical region capable of forming a trimeric coiled-coil; and
b) a N-peptide portion comprising all or a portion of the HIV gp41 NH₂-terminal heptad repeat region,
wherein the scaffold portion is fused in helical phase to the N-peptide portion, forming an α-helical domain, and wherein the three N-peptides interact with each other to form a trimeric coiled-coil.

14. The gp41 peptide mimetic of claim 13, wherein the N-peptide portion of the chimeric N-peptide is fused in helical phase to the COOH-terminus of the scaffold portion of the chimeric N-peptide.

15. The gp41 peptide mimetic of claim 13, wherein the scaffold portion of the chimeric N-peptide comprises:
a) the Suzuki-IZ coiled-coil motif (YGGIEKKIEAIEKKIEAIEKKIEAIEKKIEA (SEQ ID NO:31);
b) the IZ coiled-coil motif (IKKEIEAIKKEQEAIKKKIEAIEK (SEQ ID NO:34); or
c) the EZ coiled-coil motif (IEKKIEEIEKKIEEIEKKIEEIEK (SEQ ID NO:37).

16. A gp41 peptide mimetic which is:
a) KTA(N51)₃;
b) KTA(N51-2B)₃;
c) KTA(N51-3B)₃;
d) chA(N51)₃;
e) (CCIZN51)₃ or
f) SZN51.

17. The gp41 peptide mimetic of claim 16 which is KTA(N51)₃.

18. An immunogenic composition comprising the gp41 peptide mimetic of claim 1 and a pharmaceutically acceptable carrier.

19. A method of eliciting an immune response in a mammalian host, comprising introducing into the mammalian host a prophylatically effective amount of a immunogenic composition of claim 18.

20. The method of claim 19, wherein the mammalian host is a human.
